SEQUENCE LISTING (10 sequences)

SEQ ID NO: 1 - DNA, 29 bp, Artificial Sequence
  Description of Artificial Sequence Synthetic primer
gatgtgtacacatgacagagcagcagtgg

SEQ ID NO: 2 - DNA, 32 bp, Artificial Sequence
  Description of Artificial Sequence Synthetic primer
gatgtgtacactgagcgaacatcccagtgacg

SEQ ID NO: 3 - DNA, 29 bp, Artificial Sequence
  Description of Artificial Sequence Synthetic primer
catgtgtacacatggcagagatgaagacc

SEQ ID NO: 4 - DNA, 30 bp, Artificial Sequence
  Description of Artificial Sequence Synthetic primer
catgtgtacactgaagcccatttgcgagga

SEQ ID NO: 5 - protein, 19 aa, Artificial Sequence
  Description of Artificial Sequence Synthetic peptide
PASYMDGTMSQVGTRARLK

SEQ ID NO: 6 - protein, 9 aa, Artificial Sequence
  Description of Artificial Sequence Synthetic peptide
YMDGTMSQV

SEQ ID NO: 7 - protein, 14 aa, Artificial Sequence
  Description of Artificial Sequence Synthetic peptide
PASVLPDVFIRCGT

SEQ ID NO: 8 - protein, 9 aa, Artificial Sequence
  Description of Artificial Sequence Synthetic peptide
VLPDVFIRC

SEQ ID NO: 9 - protein, 8 aa, Artificial Sequence
  Description of Artificial Sequence Synthetic peptide
SIINFEKL

SEQ ID NO: 10 - protein, 15 aa, Artificial Sequence
  Description of Artificial Sequence Synthetic peptide
NGKLIAYPIAVEALS

(19) United States
(12) Patent Application Publication
(10) Pub. No.: US 2009/0317854 A1
(43) Pub. Date: Dec. 24, 2009

(54) Recombinant adenylate cyclase of BORDETELLA SP. for diagnostic and immunomonitoring uses, method of diagnosing or immunomonitoring using said recombinant adenylate cyclase, and kit for diagnosing or immunomonitoring comprising said recombinant adenylate cyclase

(75) Inventors: Claude Leclerc, Paris (FR); Laleh Majlessi, Montigny-Le-Bretonneux (FR); Geraldine Louf, Cachan (FR); Peter Sebo, Praha (CZ); Marcela Simsova, Vostec (CZ); Martin Vordermeier, New Haw (GB); Robert Wilkinson, Cape Town (ZA); Elisabeth Scholvinck, Groningen (NL); Jirina Loucka, Kladno (CZ)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: Institut Pasteur, INSERM (Institut National De La Sante Et De La Recherche Medicale); Universite De Prague Czech Academy Of Sciences; Veterinary Laboratories Agency-Weybridge; Imperial College London

(21) Appl. No.: 12/289,896

(22) Filed: Nov. 6, 2008

Related U.S. Application Data
(63) Continuation of application No. 11/434,892, filed on May 17, 2006, now abandoned.
Application No. 11/434,892 is a continuation of application No. PCT/EP04/14087, filed on Nov. 19, 2004.

(51) Int. Cl. C12Q 1/02

(52) U.S. Cl. 435/29

(57) ABSTRACT

Diagnostic testing and immunomonitoring that uses genetically detoxified Bordetella pertussis CyaA as a delivery system are effective in tracking any immune responses, such as those generated by infectious and non-infectious diseases, or vaccinations, for example. T cells previously stimulated by a given antigen can be restimulated in vitro by the same antigen fused or chemically coupled to CyaA or a fragment thereof. The invention includes diagnostic tests and immunomonitoring for tuberculosis by providing a delivery system, which can deliver the M. tuberculosis immunodominant proteins ESAT-6 and CFP-10, to human cells and non-human animal cells, such as cattle. In addition, fusion proteins between CyaA and cancer antigens are also provided as diagnostic tests and immunomonitoring systems for cancers, such as melanoma.

The invention relates to recombinant adenylate cyclase of Bordetella sp. for diagnostic and immunomonitoring.

### BACKGROUND OF THE INVENTION

This invention relates to diagnostic testing and immunomonitoring of diseases, as well as immunomonitoring of any T cell response following stimulation of T cells by an antigen.

The incidence of tuberculosis (TB) in cattle, caused by Mycobacterium bovis, has dramatically increased over the last decades in the British national herd. This increase constitutes a significant animal welfare, economic, and potential public health problem (Krebs et al., 1997). To control this zoonotic disease, better and more specific diagnostic reagents, as well as effective vaccines, are urgently needed. The U.K. government has initiated a research program to develop such reagents and vaccines.

Diagnosis of bovine tuberculosis in cattle is done almost exclusively in skin tests with tuberculin Purified Protein Derivative (PPD). The specificity of this test is limited because of the undefined and cross-reactive nature of PPD. A blood-based test measuring tuberculin-induced production of IFN-γ is also currently in limited field use (Wood et al., 1994). The specificity of tuberculin-based reagents is compromised, though, following vaccination with the human TB vaccine M. bovis BCG (BCG) (reviewed in Buddle et al, 2003). Therefore, diagnostic reagents allowing the differential diagnosis of M. bovis infected and vaccinated animals are needed before effective TB vaccines can be developed for cattle.

M. tuberculosis is also a major threat to human health, being responsible for more deaths globally than any other bacterium. The vaccine against, and immunological diagnosis of, TB are not fully satisfactory. For instance, the skin test reagent, PPD, used to aid diagnosis of both active and latent tuberculosis lacks specificity and sensitivity. Bacille Calmette Guerin (BCG) vaccine is very widely used to prevent TB, but its protective efficacy in adults is also limited.

Besides vaccination, an alternative control strategy to prevent the progression of latent infection by M. tuberculosis (LTBI) to clinical TB is through the use of preventative antituberculous drug therapy (PT). One aspect of this control strategy is diagnostic testing, but the tuberculin skin test (TST), used to identify healthy individuals with latent infection, has several operational drawbacks. First, the TST reagent, PPD, is cross-reactive because it contains epitopes found in many mycobacteria. TST reactivity can arise through sensitization by environmental mycobacteria or from the BCG vaccine. Second, the sensitivity of the TST is reduced by HIV infection (Johnson, J. L., et al. 1998). Third, the TST requires two clinic visits, one for administration and one for reading. The test is also operator-dependent. These limitations impair identification of LTBI and, therefore, wider application of PT. While there is a need in the art for TB vaccine candidates of greater efficacy than BCG, there is also a need for development of immunodiagnostic methods of greater sensitivity, specificity, and practicality than TST skin testing.

Previously, it has been shown that the specificity of diagnostic reagents can be improved by using antigens that are highly expressed by M. bovis or M. tuberculosis but are deleted from the genome of BCG. Such antigens allow not only the differential diagnosis of infected and BCG vaccinated animals or humans, but also improve the specificity of tuberculin per se in the absence of vaccination.

A major advance in tuberculosis research has been the identification of a genomic segment (designated Region of deletion 1—RD1) that is present in pathogenic members of the M. tuberculosis complex, but absent from all attenuated BCG strains (Gordon, S. V., et al. 1999; Behr, M. A., et al. 1999; and Mahairas, G. G., 1999). Molecules encoded on this segment can contribute to virulence (Pym, A. S., et al. 2003), or stimulate species-specific T cell responses of protective potential (Weinrich Olsen, A., et al.: 2001; Pym et al., 2003). In addition, a great deal of interest has focused on the potential of RD1 encoded antigens to improve the immunodiagnosis of TB (Arend, S. M., et al., 2000; Ewer, K., et al. 2003). However, protein subunits tend to inefficiently stimulate T cell responses and even the most promising experimental vaccine preparations require powerful adjuvants that are not licensed for use in humans. Similarly, the best immunodiagnostic methods previously known rely on peptide mixtures and ELISPOT analysis that are likely too complex for use in medically-underserved environments (Arend, S. M., et al. 2002). The antigens ESAT-6 and CFP-10, which are encoded in the RD1 region of M. bovis/M. tuberculosis, a region that is deleted in all strains of BCG, have shown particular promise as diagnostic reagents when used as recombinant proteins or synthetic peptides in the IFN-γ test (Buddle et al. 1999, Vordermeier et al., 1999 and 2001), but there is still a need in the art for simple methods by which T cell responses to M. tuberculosis antigens can be enhanced (Wilkinson, K. A., et al. 2000; Wilkinson, K. A., et al., 1999).

Under the classical pathway of antigen (Ag) presentation, exogenous and endogenous Ags of pathogens are generally processed in Ag presenting cells (APCS) by two distinct pathways to generate peptides for major histocompatibility complex (MHC)-restricted presentation (Germain, R. N. 1994). Exogenous Ags are taken up and degraded by proteases along the endocytic pathway. These processed peptides then bind nascent MHC class II molecules and are presented to CD4⁺ T cells at the APC cell membrane (Villadangos, J. 2001). After this specific recognition and interaction with co-stimulatory molecules, the activated CD4⁺ T cells can provide help to either B cells or CD8⁺ T cells by secreting cytokines. Endogenous proteins are degraded by the proteasome into the APC cytoplasm to generate MHC class I-restricted peptides that are transported to the endoplasmic reticulum where they bind to nascent MHC class I molecules. MHC I-peptide complexes are then exported and presented to CD8⁺ T cells at the APC cell membrane (Rock, K. L., and A. L. Goldberg. 1999).

In addition to these classical pathways of Ag presentation, it is now well documented that some exogenous cell-associated or particulate Ag can be cross-presented on MHC class I molecules through alternative pathways of processing (Jondal, M., et al., 1996; Heath, W. R., and F. R. Carbone. 2001; Reimann, J., and R. Schirmbeck. 1999; Moron, G., et al. 2002). One particular approach to inducing CTL responses against exogenous Ag takes advantage of the capacity of certain proteins, mainly bacterial toxins, to enter the cytosol of APC, to be processed along MHC class I presentation pathway, and to then be presented to CD8⁺ T cells. Thus, several vaccinal strategies using recombinant bacterial toxins have been designed in different laboratories to generate CTL responses against exogenous Ag (Ballard, J. D., et al. 1996; Bona, C. A. et al., 1998; Goletz, T. J., et al. 1997; Haicheur, N., et al. 2000).

An attractive approach to vaccine design is the delivery of proteins by non-replicating protein vectors such as bacterial toxins or toxoids. Bordetella pertussis secretes a calmodulin-activated adenylate cyclase toxin, CyaA, that primarily targets myeloid phagocytic cells that express the α_{M}β₂ integrin receptor (CD11b/CD18), and include professional antigen presenting cells, such as neutrophils, macrophages, NK cells, and dendritic cells (Guermonprez, P., et al., 2000). CyaA is able to deliver its N-terminal catalytic adenylate cyclase domain (400 amino acid residues) into the cytosol of eukaryotic target cells directly through the cytoplasmic membrane (Guermonprez, P., et al., 2000; Sebo, P., et al., 1995).

The CyaA is such a vector system that has shown promise in mice models. Peptide and small proteins can be inserted and expressed as fusion proteins with CyaA or chemically bound to CyaA. CyaA facilitates direct translocation across the plasma membrane of target cells. Importantly, it has been shown that vaccination with CyaA can induce MHC class I restricted CD8⁺ T cell responses (e.g. Gueromonprez et al., 1999).

Genetically detoxified CyaA can be used as a vehicle to deliver both CD4⁺ and CD8⁺ T-cell epitopes to antigen presenting cells when the epitopes are inserted within the adenylate cyclase activity domain (AC) of the CyaA toxoid in the first 600 amino acids. The antigen-presenting cells then trigger specific T cell responses (Dadaglio, G., et al., 2000; Saron, M. F., et al., 1997; Osicka, R., et al., 2000; Loucka, J., et al., 2002; Fayolle, C., et al., 1996). CyaA delivers its N-terminal catalytic domain (AC domain) into the cytosol of eukaryotic cells bearing the αₘβ₂ integrin (CD11b/CD18). CD8⁺T cell epitopes inserted into a genetically detoxified CyaA AC domain are delivered into CD11c⁺CD11b^{high} DC cytoplasm both in vitro and in vivo (Guermonprez, P., et al. 2002). This mechanism of targeted delivery of CD8⁺ T cell epitopes into MHC class I pathway results in efficient presentation followed by robust and protective CTL responses (Fayolle, C., et al. 1996, 1999, and 2001). Moreover, the T cell responses generated in vivo by this delivery system are strongly polarized toward Th1 (Dadagio, G., et al., 2000), and CD8⁺ T cells activation does not require CD4⁺ T cell help or CD40 signaling (Guermonprez, P., et al., 2002). Therefore, CyaA appears to be a safe and potent vehicle for in vivo targeted Ag delivery to CD11b^{high} DCs (El Azami El Idrissi, M., et al., 2002) leading to CD8⁺ T cell priming. Several studies have demonstrated that the generation of optimal CD8⁺ T cell responses in anti-tumoral prophylactic and therapeutic immunity, as well as against some infectious pathogens, may depend on the simultaneous activation of CD4⁺ T cells responses (Kern, D. E., et al., 1986; Toes, R. E., et al., 1999; Schnell, S., et al., 2000; Pardoll, D. M., et al., 1998; Wong, P., et al., 2003; Zajac, A. J., et al., 1998). Optimal vaccinal strategies may require the simultaneous delivery of both CD4⁺ and CD8⁺ T cell epitopes for T cell priming.

Previously, it has been shown that a MalE CD4⁺ T cell epitope inserted within the 600 first amino acids of the CyaA is efficiently targeted into MHC class II presentation pathway of APCs and presented to specific T cell hybridoma (Loucka, J., et al., 2002). Co-delivery of CD4⁺ and CD8⁺ T cell epitopes into MHC class I and class II-restricted presentation pathways, respectively, can be demonstrated with a recombinant detoxified CyaA carrying the MalE CD4⁺ T cell epitope and the OVA CD8⁺ T cell epitope in its AC domain. The capacity of this protein to deliver both epitopes for MHC-peptide complexes formation is also important.

Cattle are an ideal model to test CyaA-based constructs in an actual target species of tuberculosis. CyaA fusion proteins with mycobacterial antigens are candidates not only for subunit vaccines in cattle, but also for diagnostic antigens, particularly when they are recognized in cattle more effectively than conventional recombinant proteins.

The increased efficiency of these fusion proteins results from enhanced sensitivity because they are recognized at lower protein concentrations. The latter consideration can have major cost benefits because it can significantly reduce the amount of antigen that must be produced to implement testing, potentially by several million tests per year.

In general, there is a need in the art for diagnostic reagents for the detection of TB in animals and humans. This need exists so that differential diagnosis of M. bovis infected and vaccinated animals, such as cattle, can be made and effective TB vaccines can be developed. In humans, there is a need for the development of immunodiagnostic methods of greater sensitivity, specificity, and practicality than TST skin testing. Such immunodiagnostic methods will result from methods that allow for enhanced T cell responses to M. tuberculosis.

### BRIEF SUMMARY OF THE INVENTION

This invention aids in fulfilling the needs in the art by providing immunodiagnostic methods, especially immunodiagnostic methods carried out in vitro, that allow for enhanced T cell responses to M. tuberculosis, more particularly, this invention provides a novel system for diagnostic testing and immunomonitoring that uses genetically detoxified Bordetella sp. CyaA as a delivery system.

The invention provides methods of diagnostic testing and immunomonitoring with peptides genetically fused or chemically bound to CyaA. The results of tests with recombinant CyaA are quantitative and, therefore, can provide immunomonitoring, as well as simple diagnostic testing.

In one embodiment, the invention is a method of diagnosing or immunomonitoring a disease or immunomonitoring any T cell response following a T cell stimulation by an antigen in an animal comprising: (A) exposing a recombinant protein wherein the recombinant protein comprises a Bordetella CyaA, or a fragment thereof, and a peptide that corresponds to an antigen with which T cells of said mammal are suspected to have been previously stimulated, to a T cell of said animal; and (B) detecting a change in activation of the T cell.

In another embodiment, the invention is a kit for diagnosis or an immunomonitoring test for a disease or immunomonitoring of a T cell response following stimulation of T cells by an antigen in an animal comprising: (A) a recombinant protein wherein the recombinant protein comprises a Bordetella CyaA, or a fragment thereof, and a peptide that corresponds to an antigen with which T cells of said animal are suspected to have been previously stimulated, and (B) reagents for detecting a change in the activation of the T cell.

In embodiments of the invention, the recombinant protein comprises one or more peptides that correspond to one or more antigens.

In embodiments of the invention, the Bordetella CyaA is from Bordetella pertussis, Bordetella parapertussis, or Bordetella bronchiseptica.

In embodiments of the invention the diagnostic tests and immunomonitoring strategies can be for human or animal diseases, for example, but not limited to, cattle diseases.

In particular embodiments of the invention the disease is an infectious disease, such as tuberculosis, or is a cancer, such as melanoma.

In embodiments of the invention, the recombinant protein is CyaA-ESAT-6 or CyaA-CFP10.

In embodiments of the invention, the antigen for which the test is employed can include, but is not limited to, an infectious agent, an allergen, or an antigen from a cancer cell, such as a melanoma.

### DESCRIPTION OF THE DRAWINGS

This invention will be described in greater detail with reference to the Figures in which:

Cattle with TB Infection

FIG. 1 depicts the dose response relationship of in vitro IFN-γ production after stimulation with CFP10 (squares), and CyaA-CFP10 (triangles). The readout system was the IFN-γ ELISPOT assay. Spot forming cell (SFC) numbers from cultures with medium alone were subtracted from all values and the numbers of spots after incubation with CyaA alone were subtracted from the SFC induced by CyaA-CFP10 stimulation. Tests were performed in duplicate with 2×10⁵ PBMC/well isolated from M. bovis infected calf. Horizontal lines indicate the maximum SFC numbers (peak values) induced after stimulation with CyaA-CFP10 (a) and CFP10 (b). Line c indicates the half-maximum SFC induced after CFP-10 stimulation (50% maximum values). Vertical lines indicate the CyaA-CFP10 (d) and CFP-10 (e) concentrations required to induce 50% of CFP-10-induced peak responses (50% maximum concentration).

FIG. 2 depicts the comparison of efficacy of CyaA fusion proteins and recombinant proteins to stimulate in vitro IFN-γ production by PBMC from M. bovis infected cattle. Panel A: 50% maximum concentrations determined as illustrated in FIG. 1. Panel B: peak values determined as illustrated in FIG. 1. Readout system: IFN-γ ELISPOT assay. SFC numbers from cultures with medium alone were subtracted from all values. In addition, the numbers of spots after incubation with CyaA alone were subtracted from the SFC induced by CyaA-CFP10 stimulation. Tests were performed in duplicates with 2×10⁵ PBMC/well. A * indicates p<0.05 (two-tailed Wilcoxon signed rank matched pairs test).

FIG. 3 depicts involvement of CD11b in the recognition of CyaA-CFP10. Cultures were performed in the presence of two CD11b-specific IgG1 mAb (ILA15 and CC94). The readout system was IFN-γ ELISPOT assay. SFC numbers from cultures with medium alone were subtracted from all values. Tests were performed in duplicates with 2×10⁵ PBMC/well isolated from one infected calf. The responses are significantly different (p 0.02) for each concentration tested except the lowest concentration, as determined by the one-tailed Wilcoxon rank matched pairs test.

FIG. 4 depicts the performance of CyaA fusion proteins and recombinant ESAT-6 and CFP-10 in the whole blood BOVIGAM IFN-γ assay. Heparinized blood from 8 M. bovis infected calves was incubated with antigens at 4 nM, designated “(4)”, and 20 nM, designated “(20)”, test concentrations. IFN-γ in plasma culture supernatants was determined by ELISA. The results are expressed as OD450 units (OD450×1000). The horizontal line indicates the cut-off for positivity (100 OD450 unit). Cultures were performed in duplicate in 96 well flat-bottom plates. A * indicates p<0.05; while ** indicates p<0.01, as determined by the two-tailed Wilcoxon signed rank matched pairs test.

Human Individuals with TB Infection

FIG. 5 shows that the dose of antigen required to restimulate T cells is reduced 10-20 fold by CyaA delivery. The numbers of IFN-γ spot forming cells (SFC) were enumerated in an overnight ELISPOT assay in the presence of ESAT-6, CFP-10 or their CyaA toxoid equivalents. Concentrations shown represent the concentration of M. tuberculosis antigen. Panel A: In nine healthy TST+ve responding donors the recognition of recombinant ESAT-6 was optimal at 500 nM, whereas similar recognition occurred in the presence of 10 fold less CyaA-ESAT-6. Panel B: In ten similar donors, who responded to native CFP-10, CyaA delivery also shifted the dose response curve to the left. Approximately 10-20 times less CFP-10 was expressed as a CyaA-CFP-10 toxoid elicited the same response.

FIG. 6 shows that the detection of IFN-γ SFC in low responding subjects is enhanced by CyaA delivery. Subjects who responded to native ESAT-6 (Panel A) and/or CFP-10 (Panel B). were stratified by their magnitude of response to recombinant antigen into low (<50 IFN-γ SFC/10⁶ PBMC), intermediate (50-100) and high (>100) responders. CyaA delivery significantly increased the detection of IFN-γ SFC specifically of low responding subjects.

FIG. 7 demonstrates that both CD4⁺ and CD8⁺ responses can be enhanced by CyaA delivery. Immunomagnetic depletion of either CD4⁺ or CD8⁺ T cells from PBMC was performed and the response of the remaining cells to CyaA toxoids was assayed. The response of CD4⁺ depleted PBMC was interpreted as CD8 and vice versa. The antigen stimulated IFN-γ SFC of the CD8 depleted (CD4) was then divided by the IFN-γ SFC of the CD4 depleted (CD8) PBMC to give the CD4/CD8 ratio. The responses of individual donors are shown linked by lines. The predominant response to recombinant antigen was CD4 and CyaA delivery could enhance either CD4 or CD8 responses.

FIG. 8 shows that CD4 and CD8⁺ T cell responses to CyaA toxoids are restricted by MHC Class II and Class I molecules. The response of CD4 or CD8 depleted PBMC to CyaA toxoids was assayed in the presence or absence of inhibitors. Panels A and C: The CD8⁺ T cell response could be partially blocked by antibody to MHC Class I. Panels B and D: The CD4⁺ T cell response was sensitive to inhibition by anti-MHC Class II or chloroquine (10 mM).

FIG. 9 depicts the correlation between IFN-γ ELISPOT and whole blood assay in Panel A. The CyaA-CFP-10 stimulated overnight IFN-γ ELISPOT response was compared to the 72 hour production of IFN-γ in 1/10 diluted whole blood in 31 tuberculosis sensitized donors. Using an ELISPOT cut-off of 10 SFC/10⁶ PBMC and an ELISA cut-off of 10 pg/ml, 81% responses were concordant. The responses were also significantly correlated, using the Spearman correlation co-efficient where r=0.64, p=0.0002. Panel B depicts the enhancement of IFN-γ secretion in whole blood stimulated by CyaA carrying M. tuberculosis CFP-10. The M. tuberculosis Ag specific IFN-γ induced by ESAT-6 (Δ), CyaA-ESAT-6 (▴), CFP-10 (◯), and CyaA-CFP-10 () toxoids incorporating these antigens in a 72 hour whole blood assay was determined. Donors were then stratified into high (>1000 IFN-γ pg/ml), medium (250-1000 IFN-γ pg/ml), and low (<250 IFN-γ pg/ml), responders by their response to native antigen. The responses of low responding donors only are shown. Enhancement of the M. tuberculosis specific whole blood response by CyaA delivery of CFP-10 was significant in subjects classified as low responders to CFP-10 (p=0.021), as demonstrated by the difference in the distribution of the open and closed circles.

FIG. 10 shows that r-CyaA-ESAT-6 is able to specifically and efficiently stimulate in vitro T cells from mice infected with ESAT-6-expressing mycobacteria. Concentrations of IFN-γ produced by splenocytes of C57BL/6 mice immunized s.c. with 1×10⁶ or 1×10⁷ CFU of BCG::RD1 or BCG::pYUB412 control in response to in vitro stimulation with 10 μg/ml of various peptides, 10 μg/ml of PPD, or 2.5 μg/ml of r-CyaA. Results are expressed as the mean and standard deviation of duplicate culture wells. The label “ESAT-6(1-20)” refers to a peptide corresponding to amino acids 1-20 of ESAT-6 (immunodominant CD4+ T cell epitope). The label “MalE (10-54)” refers to a peptide corresponding to amino acids 10-54 of the MalE protein from E. coli. The label “rCyaA-OVA:257” refers to CyaA carrying an OVA CTL epitope.

FIG. 11 demonstrates that delivery of CyaA-MalE-OVA is by both MHC class I and class II pathways. As demonstrated in Panels A and B, BMDCs from C57BL/6 mice were incubated for 5 hours with various concentrations of CyaA-MalE, CyaA-OVA, CyaA-MalE-OVA, CyaA E5, MalE protein, MalE₁₀₀₋₁₁₄ or OVA₂₅₇₋₂₆₄ peptide. After incubation, BMDCs were washed and CRMC3 (Panel A and B) or B3Z T cell hybridomas (Panel C) were added to the wells. In Panel B, BMDCs were simultaneously incubated with 7.5 nM of CyaA E5 or CyaA-OVA and with various concentrations of MalE₁₀₀₋₁₁₄ peptide or protein. Five hours later, the cells were washed and 10⁵ CRMC3 T cell hybridoma were added to the wells. The culture supernatants were harvested and frozen 18 hours later. The amounts of IL-2 secreted by CRMC3 or B3Z T cell hybridomas during the culture were monitored with the IL-2 dependent CTL-L cell line as described in Example 14. The results are expressed in cpm. Each panel represents the results of at least two experiments.

FIG. 12 demonstrates that anti-CD11b mAbs block the delivery of CyaA-MalE-OVA to MHC class I and class II molecules. BMDCs were incubated with 10 μg/ml anti-CD 11b mAbs or with the same concentration of isotype control mAbs for 1 hour. Proteins or peptides (7.5 nM of CyaA-MalE-OVA and OVA p257-264 peptide and 750 nM of MalE protein) were then added to the BMDCs in the constant presence of the mAbs. APCs were washed after 4 to 5 hours of incubation with the Ags and CRMC3 (Panel A) or B3Z (Panel B) T cell hybridoma were added for 18 hours. The supernatants were tested for IL-2 content with the CTL-L cell line. The results are expressed in cpm and are representative of four experiments.

FIG. 13 demonstrates that CyaA-MalE-OVA delivery to MHC class II pathway does not require proteasome activity nor TAP transporters. Panels A and B: BMDCs were incubated for 1 hour with 3 μg/ml of lactacystin or 12 μg/ml of LLnL or LLmL. The Ags were then added and 5 hours later, the BMDCs were washed and fixed. 10⁵ CRMC3 (Panel A) and B3Z (Panel B) T cell hybridomas were then added to the wells and the culture was stopped 18 hours later. The IL-2 content in the culture supernatants was determined with CTL-L cells. Results are expressed as the percentage of residual T cell activation in the presence of the inhibitors as compared to the response obtained without inhibitors and are representative of two experiments. Panels C and D. The requirement for TAP transporters was determined with BMDCs generated from TAP1 knock-out mice. The BMDCs were incubated with various concentrations of Ags and cultured with CRMC3 (Panel C) or B3Z (Panel D) T cell hybridomas. IL-2 production by CRMC3 was determined as previously desired. The results are expressed in cpm and are representative of two experiments.

FIG. 14 demonstrates that CyaA-MalE-OVA delivery into MHC class II pathway requires endocytic protease activity and vacuolar acidification. BMDCs were incubated with leupeptin, pepstatin or graded concentrations of chloroquin (CCQ) for 1 hour and the Ags were then added to the wells at optimal concentrations (7.5 nM for CyaA-MalE-OVA, 750 nM for MalE₁₀₀₋₁₁₄ peptide and protein, 750 nM for OVA₂₅₇₋₂₆₄ peptide). After washing and fixation of the. BMDCs, CRMC3 (Panel A) or B3Z (Panel B) T cell hybridoma were added. The culture supernatants were harvested 18 hours later and their IL-2 content was determined with CTL-L. The results are expressed as the percentage of residual T cell activation as compared to the culture performed in the absence of inhibitors. They are representative of two to five experiments.

FIG. 15 demonstrates that CyaA-MalE-OVA delivery into MHC class I and class II pathways requires protein neosynthesis and Golgi transport. BMDCs were incubated for 1 hour with cycloheximide (CHX) or brefeldin A (BFA). Ags were then added (750 nM for MalE protein and peptides or 7.5 nM for CyaA-MalE-OVA). After 5 hours, the cells were washed and fixed as described in Example 18 CRMC3 (Panel A) or B3Z (Panel B) T cell hybridoma were added to the wells and the IL-2 contents in 18 hours culture supernatants was monitored with CTL-L cell line. The results are expressed in % of residual T cell activation in the presence of the inhibitors as compared to the culture performed without inhibitors and are representative of four experiments.

FIG. 16 demonstrates that MHC class II epitope delivery by CyaA-MalE-OVA does not require phagocytosis but is dependent on vacuolar acidification. For actin-dependent mechanisms inhibition, BMDCs were incubated with 10 μg/ml cytochalasin B for 1 hour at 37° C., and the Ags were added at the optimal concentrations (CyaA-MalE-OVA, OVA₂₅₇₋₂₆₄ and MalE₁₀₀₋₁₁₄ peptides at 7.5 nM, MalE protein at 750 nM). After 5 hours of incubation, BMDCs were washed three times and fixed with glutaraldehyde as detailed in Example 20. For potassium depletion, the cells were incubated in serum free medium, submitted to an hypotonic shock, and then incubated with the Ags for 45 min in the absence of K⁺ ions, as detailed in Example 20, Ags were then washed and the cells were incubated four more hours in CM and fixed. After three washes, the CRMC3 (Panel A) or B3Z (Panel B) T cell hybridoma were added at 10⁵ cell/well for 18 hours. The supernatants were tested for IL-2 content with the CTL-L cell line. For each Ag, the level of CTL-L proliferation in the absence of inhibitor was considered as the 100% of T cell activation. The results show the percentage of residual T cell activation in the presence of the drug. The results are representative of two to four experiments.

FIG. 17 demonstrates that immunization by CyaA-MalE-OVA induces both CD4⁺ and CD8⁺ T cell responses. Splenocytes of C57BL/6 mice i.v. injected with 50 μg of CyaA-MalE, CyaA-OVA, CyaA-MalE-OVA or CyaA E5 were harvested one week after immunization. (A) Splenocytes from immune mice were stimulated for 5 days in the presence of 1 μg/ml of OVA₂₅₇₋₂₆₄ peptide and tested for CTL activity on ⁵¹Cr-labeled EL-4 target cells incubated with or without the same peptide. Spontaneous cell ⁵¹Cr release was obtained with EL-4 incubated in medium alone. Each curve represents a CTL response obtained for a single mouse representative of 4 (CyaA E5) to 8 mice (CyaA-MalE, CyaA-OVA, CyaA-MalE-OVA) tested in 4 different experiments. (B, C) Splenocytes from immune mice were stimulated for 72 hours in the presence or absence of 10 μg/ml of MalE₁₀₀₋₁₁₄ peptide (B) or 1 μg/ml of OVA₂₅₇₋₂₆₄ peptide (C). The culture supernatants were tested for IL-5 and IFN-δ content in an ELISA assay. Results are expressed in pg/ml and represent the difference between the cytokine concentration in the presence and absence of the peptide. Results are representative of four experiments.

### DETAILED DESCRIPTION OF THE PREFERRED EMBODIMENTS

As shown in the Examples, T cells previously stimulated by a given antigen can be restimulated in vitro by the same antigen comprised in CyaA. Based on this discovery, the invention includes diagnostic tests and immunomonitoring for TB by providing a delivery system, which can deliver the M. tuberculosis immunodominant proteins ESAT-6 and CFP-10, as well as other proteins, as proteins comprising CyaA.

The invention also provides a simplified whole blood model to detect tuberculosis infection, wherein the frequency of positive responses to CFP-10 is increased by CyaA delivery (p=0.021). This increased frequency of positive response is an important attribute that can help identify latent infection in at risk populations, and thus facilitate better prevention of active tuberculosis.

The invention has been shown to be effective for improved diagnostic testing and immunomonitoring in animals such as cattle, as well as humans. Specifically, bovine T cells recognize CyaA fusion proteins with ESAT-6 or CFP-10 in vitro.

In addition, the invention provides for diagnostic tests and immunomonitoring for diseases other than TB. The AC domain of CyaA can co-deliver a CD8⁺ T cell epitope, OVA, and a CD4⁺ T cell epitope. MalE, into BMDCs MHC class I and class II presentation pathways, respectively. As these epitopes are not from TB, they demonstrate the utility of the non-TB embodiments of the invention. Upon CyaA delivery, there is a strong potentiation of the CD4⁺ T cell peptide presentation as compared to the MalE protein, which is abrogated by blocking CyaA interaction with its receptor by anti-CD11b mAbs. After its internalization, the AC domain is processed along either conventional endocytic routes or a cytoplasmic route to generate MalE and OVA peptides, respectively. In vivo, CyaA induces specific Th1 CD4⁺ and CD8⁺ T cell responses against MalE and OVA epitopes.

Therefore, the CyaA delivery system is useful for novel diagnostic tests because it targets DCs, delivers MHC I and II-restricted T cell epitopes for efficient presentation, and induces Th-1 polarized CD4⁺ T cell and robust CTL response in vivo.

As used herein, the term “immunomonitoring” refers to tracking the progression of or recovery from a disease with immunological assays. It refers to testing the immune responses, especially T cell responses of mammals, after stimulation by an antigen. For instance, immunomonitoring refers to testing the T cell response of vaccinated individuals, for example in clinical trials. Testing the immune response according to the invention, is especially carried out in vitro, on a biological sample. The invention is especially directed to diagnostic and immunomonitoring of tumor evolution including a tumor clearance in a human patient or in an animal, as a result of immunomonitoring of the T cell response. It is especially indicated that T cell response monitoring is in some instances of tumor immunomonitoring more appropriate than monitoring of B cell response.

As used herein, the term “antigen” refers to a heterologous peptide that can elicit an immune response. In specific embodiments, an antigen or molecule of interest is a heterologous antigen. As used herein, the term “heterologous” refers to an antigen derived from the antigen of a species other than the CyaA that is used in the vector or from an antigen of a species identical to the CyaA that is used in the vector but said antigen located in CyaA in a location where it does not naturally occur.

As used herein, the term “epitope” refers to the minimal peptide sequence of an antigen that can elicit an immune response.

As used herein, the terms “a peptide that correspond to an antigen” or “a peptide of an antigen” encompass an antigen, an epitope, or an antigen or an epitope flanked by naturally or non-naturally present flanking regions which, for example, specifically enhance antigen/epitope processing by the antigen presenting cells.

The term “restimulated” refers to the T cells of the claimed method, which were originally stimulated by the antigen upon infection, vaccination, or other exposure to antigen, especially in vivo, and are stimulated again, in vitro, in the method of the invention. The “restimulation” test according to the present invention relies on the fact that in the tested biological sample, T cells which are contacted with a determined antigen, can “respond” to this antigen (e.g., by significantly producing a cytokine, e.g., interferon) only if the patient providing the sample has previously been in contact with the agent (including infectious, tumoral or other pathogenic agent) carrying said antigen.

It has been shown in the present invention, that the recombinant protein used that comprises adenylate cyclase (CyaA) or a fragment thereof, elicits a significant increase in sensibility, to the “restimulation” test of the invention.

As used herein, the term “immunogenic” refers to a characteristic of a protein as being able to elicit an immune response.

The term “Bordetella sp. CyaA” or “Bordetella CyaA” refers to the adenylate cyclase toxoid of a pathogen of Bordetella species. Such a Bordetella CyaA can be from Bordetella pertussis, Bordetella parapertussis, or Bordetella parapertussis.

The terms “Bordetella CyaA” or “Bordetella adenylate cyclase” encompass Bordetella CyaA protein, or a fragment thereof, either modified or not, but in which the specific binding to CD11b/CD18 receptor and the process of translocation of the catalytic domain are not affected. For example, Bordetella CyaA can be modified in order to be detoxified.

The term “peptide” refers to a series of amino acids linked by amide bonds, comprising at least three amino acids and preferably more than six amino acids.

The term “tumor antigen” refers to a substance from a tumor that elicits an immune response and reacts specifically with antibodies or T cells.

The antigen portion of the recombinant protein used in the tests of the invention can be localized to any permissive site of the CyaA adenylate cyclase toxoid (see WO 93/21324). In addition, the invention encompasses tests that utilize only fragments of the CyaA adenylate cyclase in the recombinant protein (see EPO 03/291,486.3, which corresponds to U.S. Pat. Nos. 5,503,829, 5,679,784 and 5,935,580; see also El-Azami-El-Idrissi, et al., 2003, Interaction of Bordetella pertussis Adenylate Cyclase with CD11b/CD18, J. Biol. Chem., vol. 278, pp. 38514-21).

The antigen of the invention can be fused or chemically bound to CyaA (PCT/EP01/11315).

As used herein the term “fragment of the CyaA adenylate cyclase” relates to a fragment of said protein, including the CyaA protein wherein one or several amino acids which are not in the terminal parts have been deleted and the desired functional properties of the adenylate cyclase toxin are not substantially affected, i.e. the domains necessary for the specific binding to CD11b/CD18 receptor and the process of translocation of the catalytic domain are not affected. For example, a CyaA wherein the amino acids 224 to 240 have been deleted.

As used herein, the term “permissive site” relates to a site where the heterologous peptide can be inserted without substantially affecting the desired functional properties of the adenylate cyclase toxin, i.e. without affecting the domains necessary for the specific binding to CD11b/CD18 receptor and advantageously without affecting the process of translocation of the catalytic domain.

Permissive sites of the Bordetella pertussis adenylate cyclase include, but are not limited to, residues 137-138 (Val-Ala), residues 224-225 (Arg-Ala), residues 228-229 (Glu-Ala), residues 235-236 (Arg-Glu), and residues 317-318 (Ser-Ala) (see Sebo et al., 1985). The following additional permissive sites are also included in embodiments of the invention: residues 107-108 (Gly-His), residues 132-133 (Met-Ala), residues 232-233 (Gly-Leu), and 335-336 (Gly-Gln). (See generally, Glaser et al., 1988 Bordetella pertussis adenylate cyclase: the gene and the protein, Tokai J. Exp. Clin. Med., 13 Suppl.: 239-52.)

The invention encompasses diagnostic tests and immunomonitoring systems that detect any change caused by the activation of T lymphocytes. These changes include, but are not limited to changes in IL-2, IL-4, IL-5 or IFN-γ production.

The invention also encompasses diagnostic tests and immunomonitoring systems wherein the test sample can be peripheral blood mononuclear cells (PBMC), whole blood, or fractions of whole blood, for example.

The diagnostic tests and immunomonitoring systems of the invention include, but are not limited to, detection methods such as the ELISPOT assay and ELISA, or other assays using antibodies, assays using tetramers and any other assay to detect T cell activation.

Yet other embodiments of the invention include the nucleotide sequences of the inserts of the plasmids pT7CACT336/ESAT-6 and pT7CACT336/CFP-10. These plasmids were prepared as follows: The open reading frames of Mycobacterium tuberculosis H374v genes esat-6 and cfp-10 were amplified by PCR with the primers shown Table 1 and using as template the pYUB412 cosmid clone of RD1 region (Gordon, et al. 1999). The PCR product was digested by BsrG I at the sites incorporated into the PCR primers and the purified fragments encoding the antigens were inserted in-frame between codons 335 and 336 of cyaA of the pT7CACT-336-BsrG I expression vector (Osicka, et al. 2000). The exact sequence of the cloned inserts were verified by DNA sequencing. Escherichia coli XL1-Blue (Stratagene) was used throughout this work for recombinant DNA construction and for expression of antigens inserted into CyaA. Bacteria transformed with appropriate plasmids derived from pT7CACT1 (Gordon et al. 1999) were grown at 37° C. in Luria-Bertani medium supplemented with 150 μg of ampicillin per ml.

TABLE 1
PCR primers used for cloning of the esat-6
and cfp-10 genes

| Primer | sequence |
|---|---|
| Esat6-I | 5′-GATGTGTACACATGACAGAGCAGCAGTGG-3′ |
| Esat6-II | 5′-GATGTGTACACTGAGCGAACATCCCAGTGACG-3′ |
| Cfp10-I | 5′-CATGTGTACACATGGCAGAGATGAAGACC-3′ |
| Cfp10-II | 5′-CATGTGTACACTGAAGCCCATTTGCGAGGA-3′ |

Plasmid pT7CACT336/CFP-10 was deposited on Nov. 18, 2003, at C.N.C.M. under the accession number I-3135. Plasmid pT7CACT336/ESAT-6 was also deposited on Nov. 18, 2003, at C.N.C.M., Paris, France, under the accession number I-3136.

In addition, plasmid XL1/pTRACES5Tyros369, expressing CyaA-Tyr, was deposited on May 31, 2003, at C.N.C.M. under accession number I-2679. Plasmid pTRACE-5-Tyros369 is a derivative of the expression vector pTRACG that expresses the cyaC and cyaA genes from Bordetella pertussis under the control of the λ phage Pr promoter (pTRCAG also harbors an ampicillin resistance selectable marker and the thermosensitive λ repressor CI⁸⁵⁷). In pTRACE5-Tyros369, the cyaA gene is modified by insertion of a dipeptide Leu-Gln between codons 188 and 189 of wild-type CyaA (resulting in the inactivation of the adenylate cyclase activity) and by insertion of a DNA sequence encoding the following peptide sequence PASYMDGTMSQVGTRARLK inserted between codons 224 and 240 of CyaA. The underlined peptide (YMDGTMSQV) corresponds to the amino acids sequence 369-377 of tyrosinase. Plasmid XL1/pTRACES-GnTV, expressing CyaA-GnTV, was deposited on Oct. 16, 2003, at C.N.C.M., Paris, France, under accession number I-3111. Plasmid pTRACE5-GnTV is a derivative of the expression vector pTRACG that expresses the cyaC and cyaA genes from Bordetella pertussis under the control of the λ phage Pr promoter (PTRCAG also harbors an ampicillin resistance selectable marker and the thermosensitive λ repressor CI⁸⁵⁷). In pTRACE5-GnTV, the cyaA gene is modified by insertion of a dipeptide Leu-Gln between codons 188 and 189 of wild-type CyaA (resulting in the inactivation of the adenylate cyclase activity) and by insertion of a DNA sequence encoding the following peptide sequence PASVLPDVFIRCGT inserted between codons 224 and 240 of CyaA. The underlined peptide (VLPDVFIRC) corresponds to the HLA-A2 restricted melanoma epitope NA17-A derived from the N-acetylglucosaminyl-transferase V gene. (G. Dadaglio, et al. (2003) Recombinant adenylate cyclase of Bordetella pertussis induces CTL responses against HLA-A2-restricted melanoma epitope. Int. Immuno.)

Results regarding induction of a T cell response against tumoral antigens are illustrated in a publication Dadaglio G. et al (International Immunology, 2003, vol. 15, No. 12, pp. 1423-1430).

The data presented herein shows that the CyaA-CFP-10 fusion protein is recognized in vitro by bovine T cells more efficiently than CFP-10 alone, both with respect to higher maximum values and the reduced antigen concentrations needed to achieve equivalent stimulation. This recognition is CD11b-mediated. CyaA-based fusion proteins can be applied to whole blood IFN-γ tests. Both ESAT-6 and CFP-10 based CyaA fusion proteins are more strongly recognized than their non-fusion protein counterparts. CyaA-CFP10 created increased sensitivity over that created by CFP-10 alone, particularly at the lower test concentration. The Examples provided demonstrate that: CyaA fusion proteins fused to the mycobacterial antigens CFP-10 and ESAT-6 are recognized by bovine T cells and that this recognition is CD11b-mediated. These CyaA-based recombinant fusion proteins are recognized by bovine T cells more efficiently than the corresponding non-fusion proteins, allowing a reduced test concentration. The CyaA-based fusion proteins of the diagnostic tests of the invention can be applied to whole blood IFN-γ tests, and these test formats can be used in the field. The Examples show that these CyaA-based reagents are useful diagnostic reagents in cattle and subunit vaccine candidates in cattle.

Examples 3-5 show that CyaA fusion proteins are recognized in cattle via a CD11b mediated mechanism, as has been described before in the murine system. These CyaA fusion proteins that target bovine DC can also be used as subunit vaccines to induce immune responses in vivo in a similar manner, as has been described in mice. However, the unique sensitivity and specificity of ESAT-6 and CFP-10 as immuno-diagnostic reagents must be considered if they are to be used for subunit vaccination.

As demonstrated by the data shown in Examples 3-5, CyaA based fusion proteins are in vitro diagnostic reagents detecting bovine tuberculosis in cattle. The practicality of their use can be determined in large numbers of cattle with bovine tuberculosis collected from farms (field reactors), as well as cattle from herds free of bovine tuberculosis, in order to determine their sensitivity and specificity, respectively, in the field. Another determination of the practicality of those reagents in large-scale field applications is the ease with which they can be produced in large quantities and their production costs as compared to conventional recombinant proteins or synthetic peptides.

CyaA toxoids carrying ESAT-6 or CFP-10 were able to restimulate T cells from over 91.1% of TB patients and healthy sensitized donors. Delivery of antigen by CyaA decreased by 10 fold the amount of ESAT-6 and CFP-10 required to restimulate T cells and, in low responders, the overall frequency of IFN-γ producing cells detected was increased. Delivery of these antigens by CyaA enhanced the response of both CD4⁺ and CD8⁺ T cells and this response could be blocked by inhibition MHC Class II or classical MHC Class I antigen processing respectively. Antigen processing of toxoids was required as a simple mixture of CyaA carrier and ESAT-6 did not enhance the response. In addition, CD4 recognition of toxoids was sensitive to inhibition by chloroquine. In a simplified whole blood model to detect LTBI the frequency of positive responses to CFP-10 was increased by CyaA delivery, a potentially important attribute that could help identify LTBI in at risk populations, thus facilitating the better prevention of active infectious TB.

The data provided in the Examples are consistent with the interpretation that antigen delivery by CyaA increases the availability of processed M. tuberculosis derived peptide to nascent MHC molecules. It is known that CyaA toxoids become accessible to proteosomic cleavage in the cytoplasm processing as CyaA is specifically taken up via CD11b/CD18 (Guermonprez, P., et al. 2001). In vivo, CyaA has been demonstrated to be delivered efficiently to the cytosol of dendritic cells (Guermonprez, P., et al., 2001). Thus, CD8 responses are more readily detected when comparing the response to soluble recombinant antigen because it is typically processed in the endosome and, thus, less accessible to MHC Class I. CD8⁺ T cells potentially contribute to the human protective response against tuberculosis (Pathan, A. A., et al. 2000; Lalvani, A., et al., 1998), but the detection of antigen specific responses has so far been limited by the necessity to use peptide pools or recombinant Vaccinia viruses that express the antigen of interest (Pathan, A. A., et al. 2000; Lalvani, A., et al., 1998; Wilkinson, R. J., et al., 1998). Delivery of antigens by CyaA represents a novel method by which the response of CD8⁺ T cells to whole M. tuberculosis proteins can be assayed.

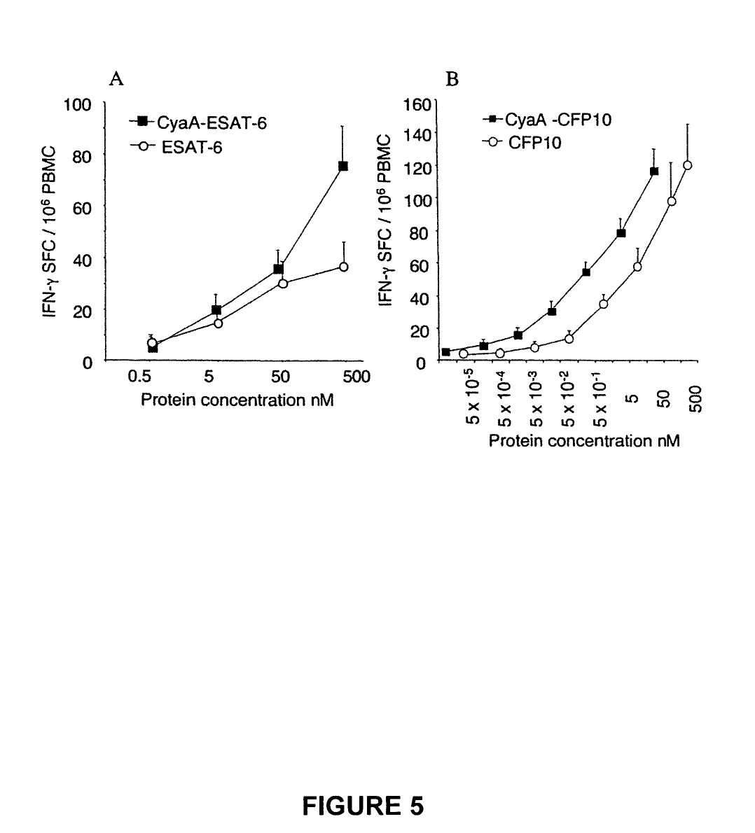

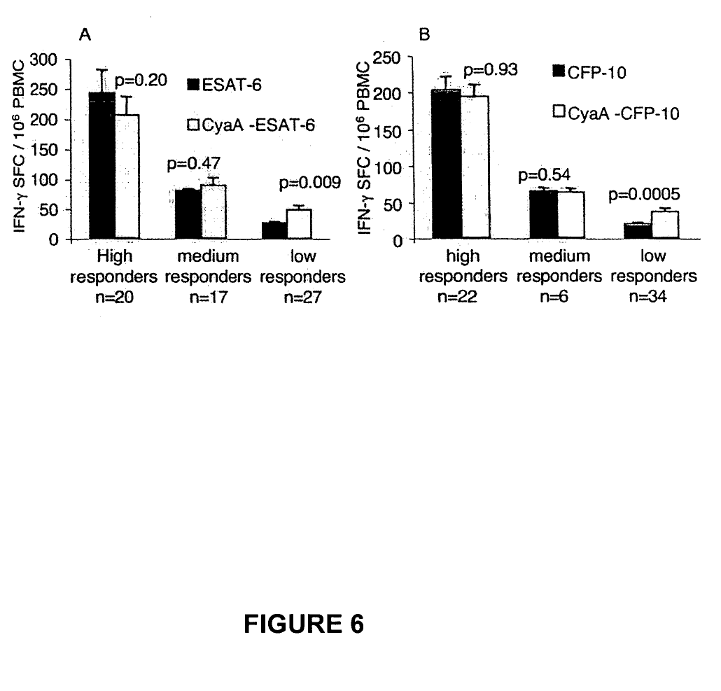

The response of M. tuberculosis specific CD4⁺ T cells was also enhanced, consistent with previous findings (Loucka, J., et al., 2002). Enhancement of the response to antigens fused to CyaA was especially pronounced in donors who have a low response to free antigen. This can be because soluble recombinant antigen is less efficiently taken up by pinocytosis (and thus less available for endosomal processing) than the macromolecular CyaA antigen conjugate that binds specifically to the CD11b/CD18 integrin receptor of antigen presenting cells (Guermonprez, P., et al., 2001) whereupon it is rapidly endocytosed (Loucka, J., et al. 2001). This would explain why on a molar basis 10-20 fold less toxoid antigen could restimulate the same response (FIG. 5) and also why some donors with negative responses to recombinant antigen did show a response to the antigen fused to CyaA (FIG. 6).

Several years ago replacement of the TST by a test that assays the in vitro production of IFN-γ produced by T cells in response to defined M. tuberculosis antigens was discussed (Jurcevic, S., et al., 1996). This approach has been refined and improved by incorporation of the highly immunogenic RD1 encoded antigens ESAT-6 and CFP-10 (Sorensen, A. L., et al., 1995; Berthet, F. X., et al., 1998). Several studies have shown that in vitro responses to RD1 encoded antigens differentiate immune sensitization by BCG from infection by pathogenic mycobacteria (Arend, S. M., et al., 2000; Cockle, P. J., et al., 2002, Lalvani, A., et al., 2001). The IFN-γ ELISPOT response to multiple peptides of ESAT-6 can be utilized to detect latent or overt tuberculosis infection with a sensitivity of 96% and a specificity of 92%. (Lalvani, A., et al., 2001). The very high frequency of recognition of the ESAT-6 and CFP-10 antigen toxoids that are observed in M. tuberculosis sensitized subjects closely accords with these estimates. The more practical approach of using antigen stimulated whole blood cultures is unfortunately associated with a fall in sensitivity to 72% (Brock, I., et al., 2001). However, the data suggest that the use of CyaA toxoid as a delivery system may overcome this deficiency. Furthermore, delivery by CyaA best enhanced the detection of IFN-γ in low responding subjects, an attribute that could be an obvious advantage in the setting of HIV.

The results of the study demonstrate that the AC domain of the CyaA is delivered in vitro into both MHC class I and class II-restricted presentation pathways, of BMDCs. A high potentiation of class II presentation by CyaA-MalE was observed as compared to the presentation of MalE protein or MalE peptide. This potentiation is dependent on CD11b-CyaA interaction as it is blocked by anti-CD11b mAbs. Using drugs and TAP1 deficient BMDCs in presentation assays, it is clear that after receptor-mediated endocytosis, the AC domain of CyaA is either translocated into the cytosol of BMDCs to be processed along conventional MHC class I processing routes or is degraded along the endocytic route of processing. In vivo, CyaA simultaneously delivers MalE and OVA peptide for CD4⁺ and CD8⁺ T cell priming and induces CTL against OVA peptide and Th-1 cytokine production specific for both MalE and OVA epitopes.

MalE CD4⁺ T cell epitope inserted into the AC domain of the genetically detoxified adenylate cyclase of Bordetella pertussis is very efficiently presented by BMDCs to CRMC3, a MalE-specific CD4⁺ T cell hybridoma. The MHC class II-restricted presentation obtained is 100 times more efficient than the presentation observed with an equivalent concentration of the purified MalE protein. It is well demonstrated that even when APCs are incubated with high concentrations of exogenous Ag, only a few MHC class II molecules present the peptides derived from that Ag (Lich, J. D., et al., 2000). Here, the potentiation of MHC class II epitope delivery by CyaA into endocytic pathway is abrogated when the interaction of CyaA with its cellular receptor CD11b is blocked by anti-CD11b mAbs. These results show that the interaction of CyaA with CD11b promotes the generation of MHC class II-restricted peptides for presentation to T cell hybridoma. The potentiation of MHC class II presentation is still observed with CyaA bearing both MalE and OVA CD8⁺ T cell epitopes. In this case, CyaA simultaneously delivers the OVA and MalE epitopes into their respective presentation pathway as efficiently as CyaAs carrying only one of these epitopes.

It has been repeatedly shown that CyaA delivers its N-terminal AC domain into target cell cytosol by a translocation that is thought to be direct and followed by AC domain processing along conventional cytosolic pathway (Ladant, D., and A. Ullmann. 1999). As CyaA AC domain is also very efficiently delivered into MHC class II presentation pathway, the processing mechanism implicated in such dual delivery was analyzed. Several studies have reported that MHC class II-restricted presentation of peptides derived from cytosolic Ags can be generated by alternative processing pathway (Rudensky, A., et al., 1991; Mukherjee, P., et al., 2001). However, the MHC class II processing of CyaA AC domain does not require proteasome activity nor TAP transporters, but is performed by endocytic proteases that are activated after vesicle acidification. These results also confirm that MalE peptide presentation requires MHC class II molecules neosynthesis. Therefore, AC domain processing for MHC class II-restricted presentation occurs along the conventional endocytic route.

This result and the CD11b requirement for CyaA presentation suggests that this toxin may enter the cell also by receptor-mediated endocytosis, followed either by the rapid translocation of AC domain from vesicles to target cell cytosol or by the degradation of this domain along endocytic vesicles. Alternatively, the AC domain is either directly translocated from cell membrane into the cytosol or taken up to enter the vesicles of the endocytic pathway. CyaA uptake does not require phagocytosis, macropinocytosis, nor caveolae-mediated endocytosis. However, the results show that MHC class II-restricted presentation of CyaA depend on CyaA internalization through a receptor-mediated endocytosis. This suggests that CD11b-mediated endocytosis is one of the mechanisms of CyaA entry into target cells. CyaA AC domain can then translocate into the cytosol to be further processed along MHC class I presentation pathway.

CyaA is a very efficient vector that targets CD11b positive cells (Guermonprez, P., et al., 2001 and 2002); and delivers peptides into MHC class I presentation pathway. This targeted delivery was shown to induce protective CTL in vivo (Fayolle, C., et al., 2001). In this study, CyaA in vivo co-delivers OVA and MalE T cell epitopes into their respective presentation pathway and induces CD8⁺ and CD4⁺ T cell responses. One injection of 50 μg of the CyaA by i.v. route, without adjuvant, induced CD4⁺ and CD8⁺ T cell responses that are polarized toward Th-1. Among CD11b⁺ cells, the CD11b CD8− DC subset is responsible for the in vivo presentation of CyaA (Guermonprez, P., et al., 2002). This murine DC subpopulation has been reported to be the most efficient in CTL induction (Schlecht, G., et al., 2001; Ruedl, C., et al., 1999) but also to bias the CD4⁺ T cell responses mostly towards Th-2. However, after activation by certain microbial compounds, this DC subset acquires the capacity to induce Th-1 T cell responses (Manickasingham, S. P., et al., 2003; Boonstra, A., et al., 2003). As shown in the Examples, the T cell responses induced are strongly polarized towards Th-1, suggesting that CyaA may promote DC maturation in addition to delivering the inserted epitopes. Additional studies can explain the nature of the signal that allows CyaA to generate Th-1 responses.

The simultaneous induction of robust Th-1 CD4⁺ and CTL CD8⁺ T cell responses is one goal of vaccination. Indeed, most of the subunits vaccines used at this time generate Th-2 polarized CD4⁺ T cell responses. In infectious diseases induced by viruses or intracellular pathogens as well as in anti-tumoral immunity, CD8⁺ T cell and Th-1 CD4⁺ T cell type of responses are required. CyaA is able to generate both CD4⁺ and CD8⁺ T cell responses very efficiently. Therefore the potentiation of MHC class II presentation observed combined with the great efficiency of CyaA in class I epitope delivery into MHC class I presentation pathway render this vector very useful for novel diagnostic tests and immunomonitoring, as well as very promising for vaccine design.

This invention will be described in greater detail in the following Examples.

### Example 1

### Materials and Methods for Bovine Studies

Bovine (PPD-B) and avian (PPD-A) tuberculin were obtained from the Tuberculin Production Unit at the Veterinary Laboratories Agency-Weybridge and used in culture at 10 μg/ml. Recombinant ESAT-6 was supplied by Dr. A. Whelan (VLA Weybridge), recombinant CFP-10 was obtained from Lionex Ltd., Braunschweig, Germany. CyaA, CyaA-CFP-10, and CyaA-ESAT-6 was provided by Dr. C. Leclerc, Institut Pasteur, Paris. Identical batches of proteins were used throughout.

M. bovis infected cattle (Vordermeier et al., 1999) Calves were infected with a M. bovis field strain from GB (AF 2122/97) by intratracheal instillation of between 5×10³ and 5×10⁴ CFU. Infection was confirmed by the presence of tuberculous lesions in the lungs and lymph nodes of these animals as well as by the culture of M. bovis from tissue collected at the postmortems performed approximately 20 weeks after the infection. Heparinized blood samples were obtained at least six weeks after infection when strong and sustained in vitro tuberculin responses were observed.

Interferon-gamma ELISPOT assay (Vordermeier et al., 2002). Peripheral blood mononuclear cells (PBMC) were isolated from heparinized blood by Histopaque-1077 (Sigma) gradient centrifugation and cultured in tissue culture medium (RPMI1640 (Life Technologies, Paisley, Scotland, U.K.) supplemented with 5% CPSR-1 (Controlled process serum replacement type-1, Sigma Aldrich, Poole, UK), non-essential amino acids (Sigma Aldrich), 5×10⁻⁵ M 2-mercaptoethanol, 100 U/ml penicillin, and 100 μg/ml streptomycin sulphate)). Direct ELISPOTs were enumerated, as described earlier. Briefly, ELISPOT plates (Immunobilon-P polyvinyldenefluoride membranes, Millipore, Molsheim, France) were coated overnight at 4° C. with the bovine IFN-γ specific monoclonal antibody 2.2.1. Unbound antibody was removed by washing and the wells were blocked with 10% FCS in RPMI1640 medium. PBMC (2-5×10⁵/well suspended in tissue culture medium (RPMI1640 supplemented with 5% CPSR-1)) were then added and cultured at 37° C. and 5% CO₂ in a humidified incubator for 24 h. Spots were developed with rabbit serum specific for IFN-γ followed by incubation with an alkaline phosphatase-conjugated monoclonal antibody specific for rabbit IgG (Sigma Aldrich). The monoclonal antibody 2.2.1 was kindly supplied by Dr. D. Godson, (Veterinary Infectious Disease Organization, Saskatoon, SK, Canada). The spots were visualized with BCIP-NBT substrate (Sigma Aldrich).

The involvement of CD11b was determined by addition (50 μl/well of ELISPOT plate) of the mouse mAb CC94 and ILA15 (both IgG1, kindly provided by Dr C. Howard, IAH, Compton, UK) to 2×105 PBMC dispensed in 100 μl. After 30 minutes pre-incubation at 37° C., serial dilutions of CyaA-CFP10 was added and the cultures incubated for 24 h as described above, followed by ELISPOT analysis.

CD4+ and CD8+ T cell subpopulations were depleted by magnetic negative selection using the ant-bovine CD4 or CD8 specific mAb CC30 and CC58 (C. Howard, IAH) in conjunction with the MACS system (goat anti-mouse IgG coated beads, LS separation columns, Miltenyi Biotec Ltd, Bergisch-Gladbach, Germany) as described earlier (Vordermeier et al., 2001).

Interferon-gamma (IFN-γ) assay (Wood et al., 1994; Vordermeier et al., 1999). Whole blood cultures were performed in 96-well plates in 0.2 ml/well aliquots by mixing 0.1 ml of heparinized blood with an equal volume of antigen containing-solution. Supernatants were harvested after 24 h of culture and interferon-gamma (IFN-γ) determined using the Bovigam EIA kit (CSL, Melbourne, Australia) (Vordermeier et al., 2002). The data are expressed as OD450 units (OD450×1000). CyaA background levels were subtracted from CyaA-ESAT6 and CyaA-CFP10 values.

Statistical analysis. Statistical analysis was performed using Instat v3.0a (GraphPad, San Diego, Calif., USA) on an iMac personal computer. Data were analyzed using the one- or two-tailed Wilcoxon signed rank matched pairs test. See figure legends for further details.

### Example 2

### Construction and Purification of Recombinant CyaA Carrying Entire Mycobacterial Antigens Cfp10 or Esat-6

Escherichia coli XL1-Blue (Stratagene) was used for recombinant DNA construction and for expression of antigens inserted into CyaA. Bacteria transformed with appropriate plasmids derived from pT7CACT1 (Osicka et al., 2000) were grown at 37° C. in Luria-Bertani medium supplemented with 150 μg of ampicillin per ml. The open reading frames of Mycobacterium tuberculosis H37Rv genes esat-6 and cfp-10 were amplified by PCR from the pYUB412 cosmid clone of the RD1 region (Gordon et al., 1999) using the following primers:

| | |
|---|---|
| Esat6-I | 5′-GATGTGTACACATGACAGAGCAGCAGTGG-3′ |
| Esat6-II | 5′-GATGTGTACACTGAGCGAACATCCCAGTGACG-3′ |
| CFP-10-I | 5′-CATGTGTACACATGGCAGAGATGAAGACC-3′ |
| CFP-10-II | 5′-CATGTGTACACTGAAGCCCATTTGCGAGGA-3′. |

The PCR product was digested by BsrG I at the sites incorporated into the PCR primers and the purified fragments encoding the antigens were inserted in-frame between codons 335 and 336 of the cyaA gene open reading frame born on the pT7CACT-336-BsrG I expression vector (Osicka et al., 2000). The exact sequence of the cloned inserts was verified by DNA sequencing.

The control detoxified mock CyaA and the recombinant CyaA proteins carrying the ESAT-6 and CFP-10 antigens, respectively, were produced in E. coli, purified from inclusion bodies by a combination of ion-exchange chromatography on DEAE-sepharose and hydrophobic chromatography on Phenyl-sepharose, as described previously (Karimova et al., 1998). In the final step, the proteins were eluted with 8 M urea, 50 mM Tris-Cl pH 8, 2 mM EDTA and characterized as previously described (Karimova et al., 1998). The resulting proteins were free of any detectable adenylate cyclase enzymatic activity.

### Example 3

### IFN-γ Responses of Experimentally Infected Cattle

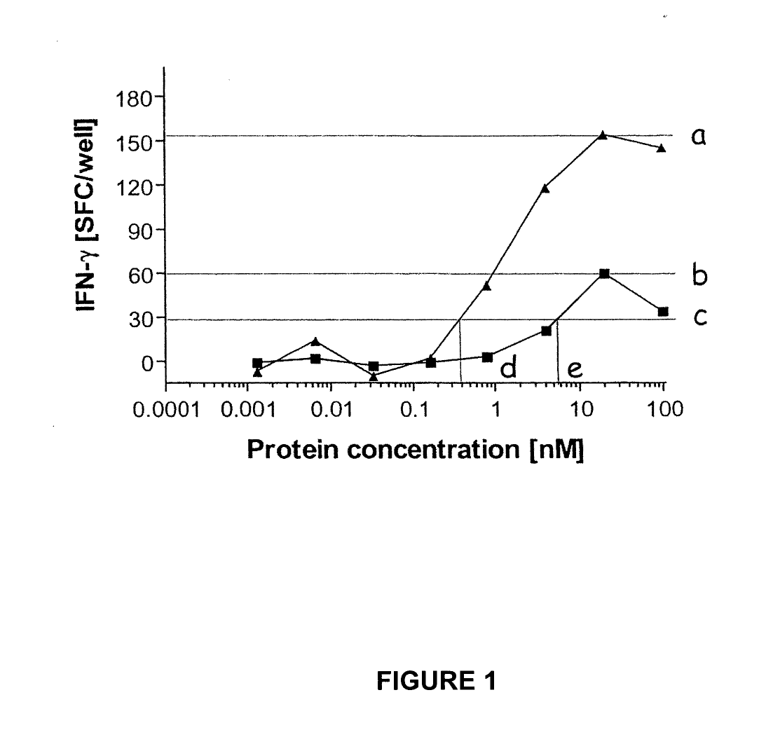

PBMC were prepared from experimentally infected cattle and incubated with serial dilutions of antigens (recombinant ESAT-6, CFP-10, CyaA-ESAT6, CyaA-CFP10, and CyaA control). The antigen-induced IFN-γ responses were determined after 24 h culture using a sensitive ELISPOT assay. The number of spot-forming cells (SFC) found without antigen added (medium controls) were subtracted, the number of SFC obtained after CyaA stimulation were subtracted from the number of SFC induced after CyaA-ESAT6 and CyaA-CFP10 stimulation. To illustrate how the data were subsequently expressed and compared, a representative result for CFP-10 tested in one calf is given in FIG. 1. In this calf, CyaA-CFP-10 induced both a higher peak response than recombinant CFP-10 (as shown by comparison of values indicated by horizontal lines a and b), and was recognized more effectively as indicated by the vertical lines d and e, which indicate the concentrations required for ‘half-maximum’ (50% of peak responses) responses induced with the recombinant protein (line c).

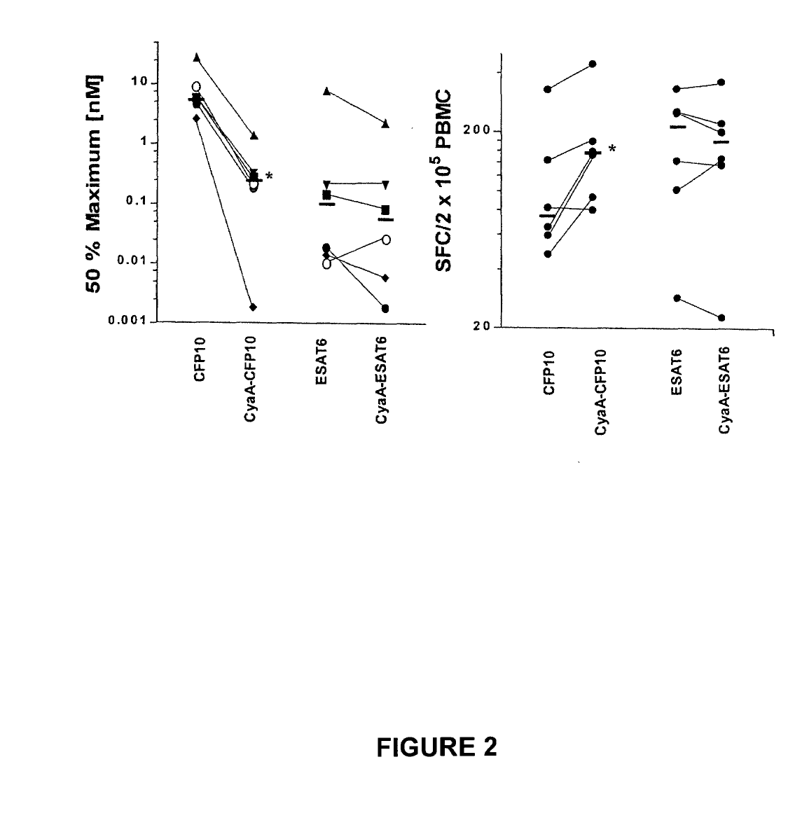

Subsequently a further batch of six experimentally M. bovis infected calves were tested and the results interpreted identically. As demonstrated by the comparison of peak responses induced by CFP-10 or CyaA-CFP10, and the reduced concentration needed for 50% maximal responses, the CyaA-CFP10 fusion protein was superior to its non-fusion counterpart (FIG. 2): CyaA-CFP10 peak responses were about twice as high as those observed with CFP-10 (median responses; CyaA-CFP10: 157 SFC; CFP-10: 75 SFC; p=0.03). As judged by the concentrations required for 50% maximum responses, CyaA-CFP10 was recognized about 20 times more efficiently than CFP-10 (50% maximum concentrations; CyaA-CFP10: 0.3 nM; CFP-10: 6.25 nM, p=0.017).

The IFN-γ responses induced by ESAT-6 or the CyaA-Esat-6 fusion proteins were not significantly different from each other (FIG. 2). Interestingly, recombinant ESAT-6 was about 70 times more efficiently recognized than CFP-10 (median of 50% maximum concentrations: 0.09 with ESAT-6 compared to 6.25 with CFP-10). This difference in the efficiency between those two proteins might explain why an additional benefit of presenting ESAT-6 as a CyaA fusion protein was not realized in these experiments.

### Example 4

### Recognition of CyaA-CFP10 is Mediated by CD11b

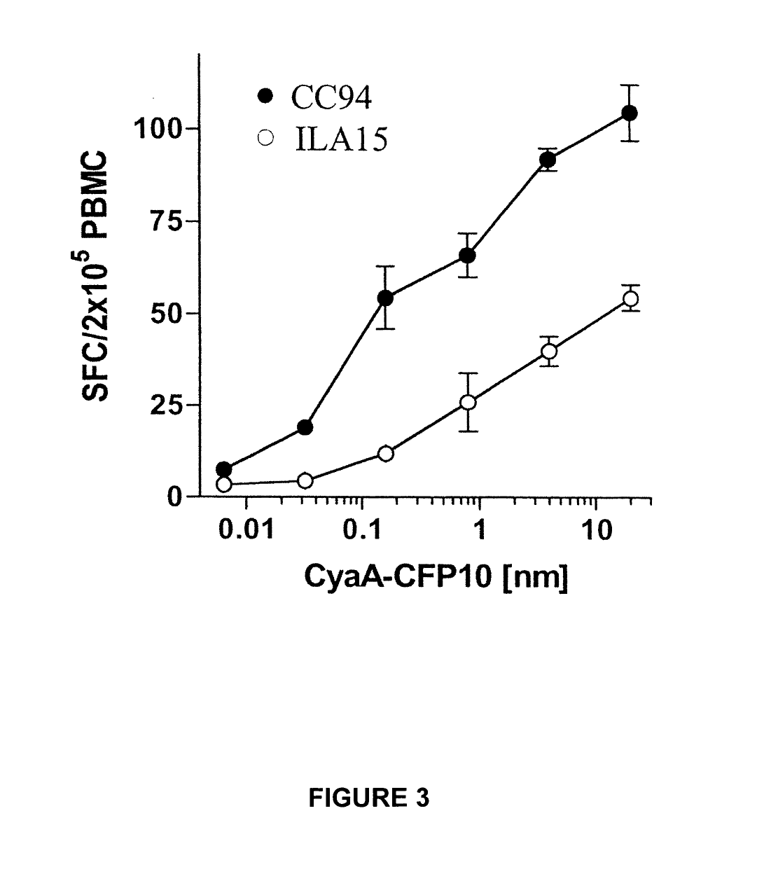

To determine whether the recognition of CyaA-CFP10 is mediated via a CD11b-dependent mechanism (as has been recently shown for mice), PBMC from an infected calf were stimulated with CyaA-CFP10 in the presence of two mAb of the same isotype (IgG1) specific for bovine CD11b (kindly provided by Dr C. Howard, IAH, Compton, UK). One of these mAb, (ILA15) interfered with the interaction of CyaA-CFP10 with CD11b as the number of SFC was reduced significantly, whereas the non-blocking isotype control mAb (CC94), did not (FIG. 3, p<0.02 for each concentration tested as determined by the one-tailed Wilcoxon matched pairs test). These results provide evidence that, as in the murine system, CyaA interacts with CD11b on APC in cattle.

CyaA-CFP10 were also -shown to be recognized by both CD4+ and CD8+ T cells. This was analyzed by depleting either sub-population with magnetic beads. Cattle at early stages of bovine tuberculosis display only weak or undetectable CD8+ T cell responses (Pollock et al., 1996, Vordermeier, unpublished observation). Consequently, all of the experimentally infected animals available for this study were tested relatively early following infection (i.e. approximately 4-6 months post-infection), and significant PPD-B and CFP-10-specific CD8+ T cell responses were observed in only in one of four cows tested. Nevertheless, the results obtained from the adult cow that was infected several years previously, indicated that CyaA-CFP10 was recognized by CD4+ and CD8+ T cells, as was the recombinant protein. However, CyaA-CFP10 induced higher in vitro CD8+ T cell responses compared to the recombinant protein (CD8/CD4 ratio of responding cells: CFP-10: 0.8, CyaA-CFP10: 1.05, data not shown), though this difference was not statistically significant.

### Example 5

### Performance of CyaA-ESAT-6 and CyaA-CFP10 in Whole Blood IFN-γ Tests (BOVIGAM Assay)

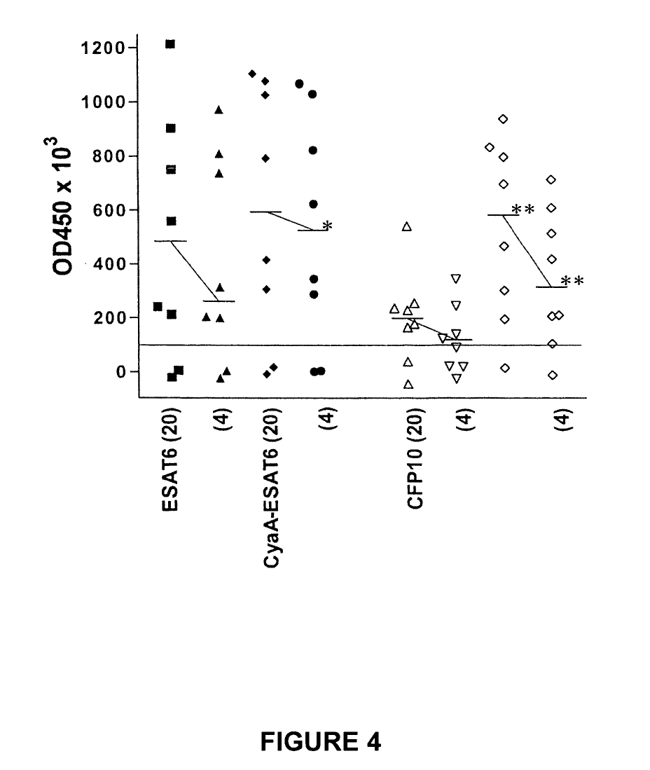

The IFN-γ test was applied as diagnostic assay in the field in the format of a whole blood assay (BOVIGAM test). In this format, blood collected on farms was heparinized and incubated with either tuberculins or specific antigens. After a 24 h incubation period, the amount of antigen-induced IFN-γ in plasma supernatants was determined by ELISA. To determine the performance of the CyaA fusion proteins with ESAT-6 and CFP-10, blood was obtained from a second batch of eight experimentally M. bovis infected calves. These blood samples were stimulated with ESAT-6, CFP-10, CyaA-ESAT6, and CyaA-CFP10 at 4 and 20 nM concentrations. The results of the ELISA assay conducted 24 h later are shown in FIG. 4. As shown above for PBMC responses measured by ELISPOT, significantly stronger IFN-γ responses were observed with CyaA-CFP10 at both test concentrations compared to recombinant CFP-10 protein (p=0.078 at both concentrations). This increased response was particularly evident when the blood was stimulated with antigen at 4 nM concentration (median OD450 units with CyaA-CFP10: 313, with CFP-10: 105). While the responses between CyaA-ESAT6 and ESAT6 were not significantly different at 20 nM, significantly elevated responses were observed after stimulation with CyaA-ESAT-6 at 4 nM (p=0.015, median OD450 units with CyaA-ESAT6: 486; with ESAT-6: 260).

When the diagnostic outcome was evaluated using the commonly applied cut-off of 100 OD450 units, six of eight tested animals were deemed positive for bovine TB using ESAT-6 and CyaA-ESAT6 applied at both test concentrations (FIG. 4). In contrast, the use of CyaA-CFP10 improved the sensitivity of CFP-10 as antigen, because seven of eight and six of eight of the animals tested positive at 20 and 4 nM test concentration with CyaA-CFP10, whereas six of eight and four of eight were classified positive after stimulation with recombinant CFP-10 at corresponding test concentrations (FIG. 4).

One of the eight test negative animals presented without tuberculous lesions at a post-mortem carried out several months after this experiment was performed, though M. bovis could be cultured from any tissue samples taken. This animal was also tuberculin skin test negative. Taken together, this suggests that the experimental infection in this animal was contained and did not result in disease. As expected, no IFN-γ was induced in the blood of this calf after stimulation with either PPD-B, CyaA-ESAT-6, CyaA-CFP10, ESAT-6, or CFP-10, thus highlighting the specificity of these reagents (FIG. 4).

### Example 6

### Materials and Methods for Human Studies

Human studies were conducted with ethical approval from the Harrow Local Research Ethics Committee (Harrow LREC 1646 and 2414). Patients with tuberculosis and their healthy contacts were recruited from Northwick Park Hospital, Harrow (North West London Hospitals NHS Trust). Three groups of people with distinct clinical phenotypes were selected. The first group was adults with overt (i.e. culture or biopsy positive) tuberculosis (n=21, 14 M, 7 F, average age 35.1 years). The second group consisted of asymptomatic adults with normal chest radiographs who nevertheless exhibited strongly positive TST reactions (Heaf Grade 3 and above) and were thus thought likely to have LTBI (n=44, 26M, 18F, average age 34.7 years). A third control group consisted of healthy adults with no documented exposure to TB and whose skin test reactions were negative (n=7, 3M, 4F, average age 37.6 years). The first two groups were chosen to maximize the chances of T cell reactivity to M. tuberculosis specific antigens and thus allow the comparison of response to recombinant and CyaA toxoids. All subjects were subsequently advised and, if indicated, treated according to British Thoracic Society guidelines (see Thorax 55:887-901, 2000).

Cells. PBMC were separated from 20 mls of blood by centrifugation over Ficoll-Paque Plus (Pharmacia, Uppsala, Sweden), and suspended in RPMI supplemented with 2 mM L-glutamine, penicillin 100 U/ml, gentamicin 5 μg/ml and 10% heat-inactivated fetal calf serum (Sigma, St. Louis, Mo.) (R10). CD4⁺ and CD8⁺ T cells were depleted using anti-CD4 or anti-CD8 mAb conjugated to ferrous beads (Dynabeads M-450, Dynal, Oslo, Norway) according to the manufacturer's instructions. These depletions consistently yielded cells populations with 97-99% purity. Anti MHC Class II blocking antibody (L243, Leinco Technologies), anti MHC Class I blocking antibody (W6/32, Leinco) and isotype control antibody (Mouse IgG2a, Leinco) were used at 5 μg/ml 30 minutes after addition of antigens. Chloroquine (Sigma) at 10 μg/ml was added to the cultures just before the antigens.

Ex vivo enzyme-linked immunospot (ELISPOT) assay for single cell IFN-γ release. 96-well PVDF-backed plates (MAIPS45, Millipore, Bedford, Mass.), pre-coated with 15 μg/ml of anti-IFN-γ mAb 1-DIK (Mabtech, Nacka, Sweden), were blocked with R10 for 2 hrs. 3×10⁵ PBMC were added in 100 μl R10/well. Duplicate wells of CyaA toxoids and recombinant ESAT-6 and CFP10 were used at the optimum concentrations derived from FIG. 5. PPD (Evans Medical, Liverpool, UK) at 100 U/ml, and phytohaemagglutinin (ICN Biomedicals, Aurora, Ohio) at 5 μg/ml was added to positive control wells. No antigen was added to the negative control wells. After 14 h incubation at 37° C. in 5% CO₂, plates were washed with PBS containing 0.05% Tween-20. 50 μl of 1 μg/ml of biotinylated anti-IFN-γ mAb, 7-B6-1-biotin (Mabtech), was added for 2 h. Plates were washed and streptavidin-alkaline phosphatase toxoid (Mabtech) was added at 1:1000 dilution. After 1 h and further washing, 50 μl of chromogenic alkaline phosphatase substrate (Biorad, Hercules, Calif., USA), diluted 1:25 with deionized water, was added. Ten minutes later the plates were washed and allowed to dry, and spot forming cells (SFC) were enumerated with a magnifying glass.

Recombinant antigen and CyaA toxoid construction. Recombinant native ESAT-6 was prepared as previously described and was a gift from the Veterinary Laboratories Agency, Weybridge, Surrey KT15, UK. Recombinant CFP-10 was obtained commercially from Lionex (Braunschweig, Germany). N-terminal sequencing confirmed the identity of the cloned antigen. Escherichia coli XL1-Blue (Stratagene) was used throughout this work for recombinant DNA construction and for expression of antigens inserted into CyaA. Bacteria transformed with appropriate plasmids derived from pT7CACT1 (Osicka, R., 2000) were grown at 37° C. in Luria-Bertani medium supplemented with 150 μg of ampicillin per ml. The open reading frames of Mycobacterium tuberculosis H37Rv genes esat-6 and cfp-10 were amplified by PCR from the pYUB412 cosmid clone of the RD1 region (Gordon, S. V., et al., 1999) using the following primers:

| | |
|---|---|
| Esat6-I | 5′-GATGTGTACACATGACAGAGCAGCAGTGG-3′ |
| Esat6-II | 5′-GATGTGTACACTGAGCGAACATCCCAGTGACG-3′ |
| CFP-10-I | 5′-CATGTGTACACATGGCAGAGATGAAGACC-3′ |
| CFP-10-II | 5′-CATGTGTACACTGAAGCCCATTTGCGAGGA-3′ |

The PCR product was digested by BsrG I at the sites incorporated into the PCR primers and the purified fragments encoding the antigens were inserted in-frame between codons 335 and 336 of CyaA on the pT7CACT-336-BsrG I expression vector (Osicka, et al., 2000). The exact sequence of the cloned inserts was verified by DNA sequencing.

The control detoxified mock CyaA and the recombinant CyaA proteins carrying the ESAT-6 and CFP-10 antigens, respectively, were produced in E. coli, purified from inclusion bodies in 8 M urea, 50 mM Tris-Cl pH 8, 2 mM EDTA and characterized as previously described (Sebo, et al., 1999). The resulting proteins were free of any detectable adenylate cyclase enzymatic activity.

Whole blood assay and Interferon-γ ELISA. Venous blood was collected (BD Na Heparin vacutainer, Cat 368480) and processed within 4 hours of sampling. Whole blood was diluted 1:10 in RPMI (supplemented with glutamine and penicillin/streptomycin). 180 μl of the diluted blood was plated in 96-welled round-bottomed plates with stimulating antigens in duplicate wells. The final concentrations of the antigens were: 250 nM (rESAT-6), 50 nM (CyaA-ESAT-6), 500 nM (rCFP-10), 50 nM (CyaA -CFP-10), 50 nM (mock CyaA toxoid), 5 μg/ml (PHA: positive control) and 20 μl/ml (RPMI: negative control). Stimulated whole blood was cultured at 37° C. in a CO₂ incubator. Supernatants from duplicate wells were harvested after 60-72 hours of culture, pooled and immediately frozen for later IFN-γ measurements by ELISA. The optimal concentrations of stimulants and the timing of harvesting had been previously determined by dose-response and time-course experiments. ELISA reactions were performed in accordance with antibody manufacturers' instructions. Briefly, 96-welled flat-bottomed plates were coated overnight at 4° C. with purified mouse anti-human IFN-γ (BD Pharmigen 554548). After blocking and washing, wells were plated with supernatants (1:2 dilutions in duplicate) and standards (standard curve dilutions from 15 pg/ml-10 000 pg/ml, duplicate measurements) after which the plates were again incubated at 4° C. overnight. After washing, the wells were incubated with biotinylated mouse anti-human IFN-γ (BD Pharmingen, 554550) for 1.5 hours at room temperature, washed again and incubated with Streptavidin (Sigma Cat no A3151) for 30 mins. OPD was used as a substrate for detection and 2N H₂SO₄ to stop color development. Optical densities were read at 490 nm on a plate reader and IFN-γ concentrations were calculated from standard curves. The Spearman rank correlation coefficients between independent variables were calculated using SPSS-10.

### Example 7

### The Dose of ESAT-6 or CFP-10 Required to Restimulate M. tuberculosis Specific T Cells is Reduced 10-20 Fold by CyaA Delivery

The optimum stimulatory dose of ESAT-6 and CFP-10 and the respective CyaA toxoids in vitro was determined, using the equivalent dose of antigen inserted in the recombinant molecule (i.e. the same molar amount of protein) as the CyaA toxoid. The numbers of IFN-γ SFC were then enumerated in an overnight ELISPOT assay. These experiments with CyaA toxoids were controlled by subtracting the number of IFN-γ SFC in wells containing the same amount of CyaA toxoid into which no antigenic stimulus had been inserted (mock toxoid). In nine healthy TST+ve donors who responded to ESAT-6, optimal recognition of this molecule was observed at a dose of 500 nM. Ten fold less ESAT-6 (50 nM) was required when the antigen was presented as CyaA-ESAT-6 (FIG. 5A).

Interestingly, increasing the dose of CyaA-ESAT-6 to 500 nM indicated that delivery by CyaA vector could lead to an overall increase in IFN-γ SFC detected. However, further increase in the dose of CyaA toxoid was associated with a decrease in SFC that was due the high urea content of solutions necessary to solubilize the CyaA toxoid (data not shown). In ten similar donors who responded to rCFP-10, CyaA fusion similarly shifted the dose response curve to the left. Approximately 10-20 times less CFP-10 expressed as a CyaA toxoid elicited the same response as native antigen (FIG. 5B). Thus, CyaA fusion decreased by ten fold the amount of ESAT-6 and CFP-10 required to restimulate T cells. In addition, there was also the potential to increase the overall number of IFN-γ SFC detected, particularly for ESAT-6.

### Example 8

### The Detection of IFN-γ SFC in Low Responding Subjects is Enhanced by CyaA Delivery

Based on the results shown in FIG. 5 500 nM rESAT-6 and 50 nM for the CyaA-ESAT-6, and 250 nM of CFP-10 and 25 nM CyaA-CFP-10 were selected for further experimentation, on the basis of equivalent potency. To determine whether delivery by CyaA would lead to enhancement of the number of IFN-γ SFC, larger group of patients and healthy sensitized subjects was studied. There was no difference in the frequency or magnitude of responses to any stimulus between the patient and healthy sensitized subjects and so results were combined for analysis. Sixty-three of sixty-eight donors responded (>10 SFC/10⁶ PBMC) to rESAT-6 (average IFN-γ SFC/million PBMC was 107.7±16.2) and 64 to CyaA-ESAT-6 (107.5±12.9); 52 responded to rCFP-10 (104.4±14.3) and 62 to CyaA-CFP-10 (94.9±11.3). Thus no overall difference between the frequencies of IFN-γ SFC following stimulation of PBMC with the recombinant or toxoids was apparent. However, the proportion of subjects responding to CFP-10 increased from 76.4 to 91.1%.

When the subjects' responses were analyzed according to their response to the recombinant antigens into low (<50 IFN-γ SFC/10⁶ PBMC), intermediate (>50<100 SFC/10⁶ PBMC) or high responders (>100 SFC/10⁶ PBMC), clear enhancement in the group of low responders was seen. Thus, the average number of detected IFN-γ SFC/million increased from 26.1±2.7 to 48.2±7.3 (n=27, p=0.009) for ESAT-6 and from 17.5±2.6 to 36.8±4.3 (n=34, p=0.0002) in the case of CFP-10 (FIG. 6). Interestingly, the PBMC of ten donors who did not respond to rCFP-10 did produce IFN-γ following stimulation with CyaA-CFP-10 (mean IFN-γ SFC/million 37.8±8.2). This indicates that the overall number of CFP-10 specific IFN-γ SFC detected could also be increased as was originally seen for ESAT-6 (FIG. 5).

### Example 9

### Both CD4⁺ and CD8⁺ Responses can be Enhanced by CyaA Delivery

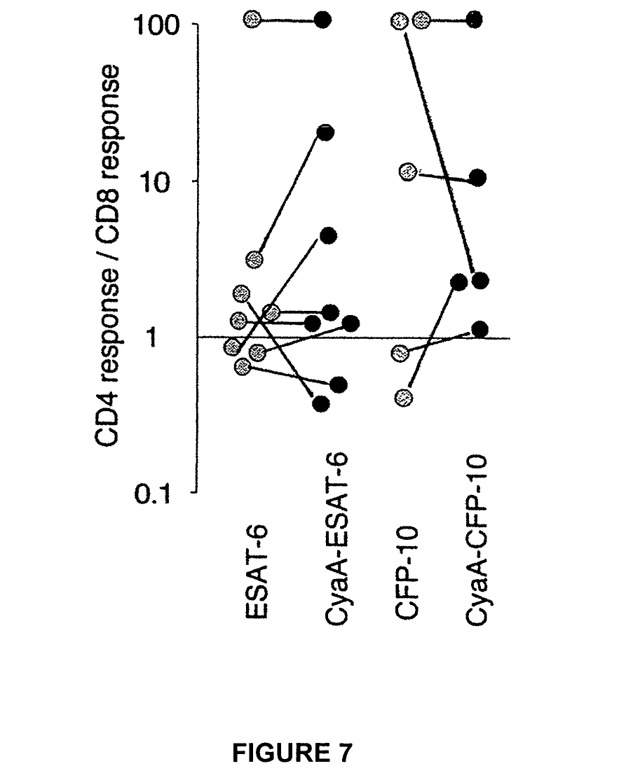

In order to define the T cell subset that recognized the CyaA toxoids, populations were enriched by performing prior immunomagnetic depletion of either CD4⁺ or CD8⁺ T cells from PBMC. The remaining cells were set up in the ELISPOT assays and stimulated overnight with the ESAT-6 or CFP-10 and detoxified CyaA incorporating the same antigens. Eight donors were tested for the CyaA-ESAT-6 and five donors for the CyaA-CFP-10 and the corresponding recombinant antigens. Both CD4⁺ and CD8⁺ responses were seen to the recombinant antigens, the CD4⁺ response being dominant (FIG. 7). When compared to the response to recombinant antigen, responses to the CyaA toxoids clearly shifted towards CD4 in three instances, and towards CD8 in two (FIG. 7). There was no net change in the remaining eight cases.

### Example 10

### The Enhanced Detection of IFN-γ SFC Requires Covalent Association of the Antigen to CyaA, which Must be Processed for Presentation Via the MHC

To determine whether the M. tuberculosis protein has to be covalently linked to CyaA, the effect of mixing ESAT-6 with mock CyaA toxoid was tested. PBMC from four subjects were set up with ESAT-6 (500 nM), CyaA-ESAT-6 (50 nM) or the mixture of rESAT-6 (500 nM) and CyaA (50 nM). The median IFN-γ SFC/million for these stimulants was 113, 147 and 58 respectively, showing that covalent linkage between the antigen and carrier is required for enhancement to occur (data not shown). In fact, it appeared that simple mixture of rESAT-6 with CyaA might have actually decreased the response to rESAT-6.

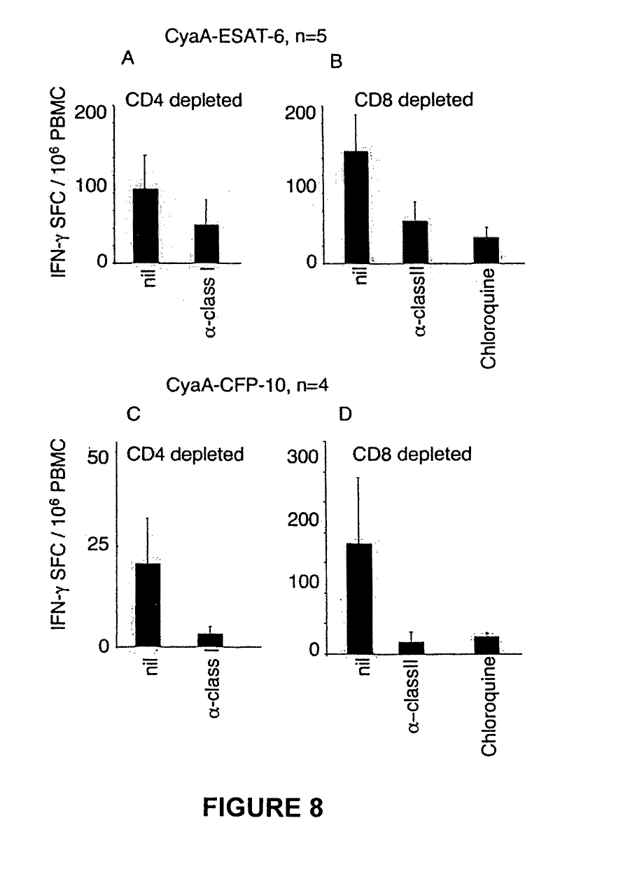

Next, whether the CD4⁺ and CD8⁺ T cells that recognized CyaA toxoid were classically MHC Class I or Class II restricted was determined. CD4 or CD8 depleted cells were set up on the ELISPOT plates and stimulated with CyaA-ESAT-6 (5 donors) or CyaA-CFP-10 (4 donors). Anti MHC Class I blocking antibody, anti MHC Class II antibody, or isotype control (all at 5 μg/ml) was added to selected wells and the plates were incubated overnight. The IFN-γ response of CD4 depleted (interpreted as CD8) T cells in response to CyaA-ESAT-6 and CyaA-CFP-10 was 48% and 83% inhibited by anti MHC Class I antibody respectively (FIGS. 8A and C). The CD8 depleted (interpreted as CD4) T cell response was 62% and 88% inhibited by anti MHC Class II antibody (FIGS. 8B and D). Isotype control antibodies had no effect on recognition (data not shown). In addition, chloroquine inhibited by 77% and 84% the CD4 response to CyaA-ESAT-6 and CyaA-CFP-10 toxoids respectively (FIGS. 4B and D). Taken together these data show that the response to M. tuberculosis antigens delivered as CyaA toxoids require antigen processing, and that the MHC recognition of the inserted M. tuberculosis molecules is classically restricted.

### Example 11

### The Response to CyaA-CFP-10 is Also Enhanced in a Simple Whole Blood IFN-γ Production Assay

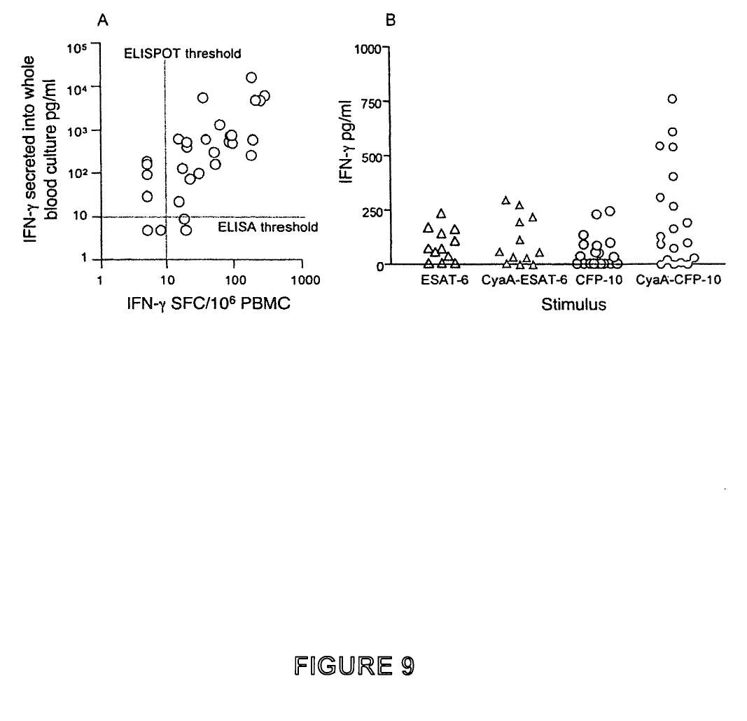

Detection of IFN-γ secreted into the supernatant of whole blood cultures requires less blood and is potentially more applicable to field conditions than the ex-vivo IFN-γ ELISPOT assay. However, it appears that such whole blood assays, while retaining specificity, are less sensitive than ELISPOT detection. Therefore, it was examined whether the enhanced response to CyaA toxoids carrying ESAT-6 or CFP-10 could compensate this deficiency. Thirty-three patients and healthy sensitized subjects were tested in parallel using the-two read-out assays for IFN-γ production. All 33 donors responded to rESAT-6, and 31 donors responded to rCFP-10. The ELISPOT and whole blood IFN-γ responses to CyaA-ESAT-6 and CyaA-CFP-10 were positively correlated (r=0.58 and 0.64 respectively, p<0.001 in both cases, FIG. 9A). Donors were stratified according to their responses to the free antigen into low (<250 μg/ml IFN-γ), intermediate (250-1000 μg/ml IFN-γ), or high responders (>1000 μg/ml IFN-γ) in the whole blood assay. The results showed a similar effect of CyaA delivery on antigen recognition as found by the ELISPOT assay. Thus, in low responding subjects the amount of IFN-γ produced in the presence of CyaA-CFP-10 was on average of 27.7±9.5 fold higher than in the presence of free rCFP-10 (p=0.021, FIG. 9B). The response to CyaA-ESAT-6 showed the same general trend (average 5.6±3.2 fold) although this effect did not reach statistical significance.

### Example 12

### Specific and Efficient in Vitro Stimulation of Mycobacteria-Primed T-Cells with r-CyaA-ESAT-6

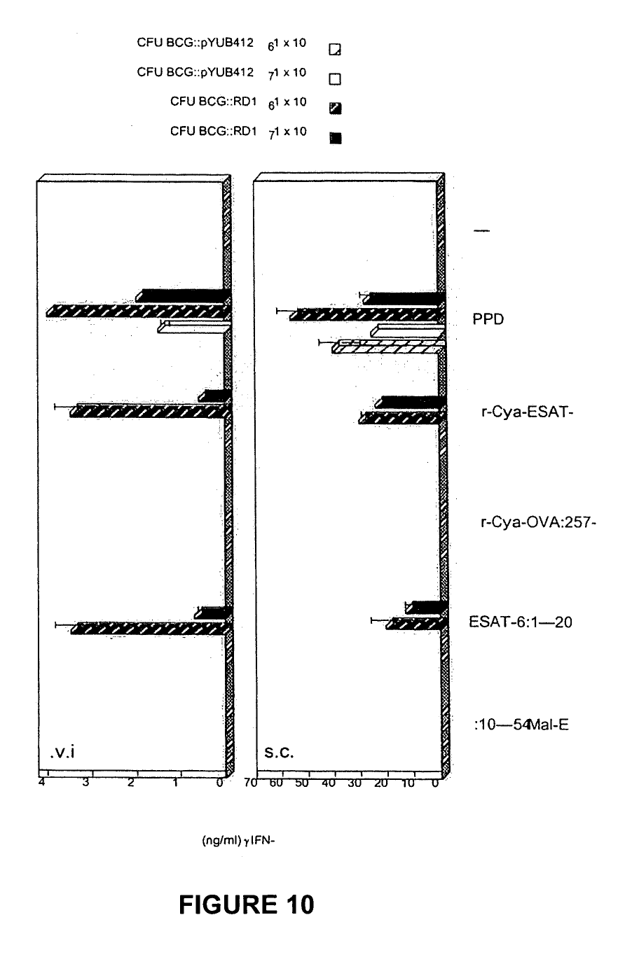

Splenocytes of C57BL/6 mice infected (s.c. or i.v.) with 1×10⁶ or 1×10⁷ CFU of a BCG strain, stably complemented with the RD1 chromosomal region of M. tuberculosis (referred to as BCG::RD1) (Pym, 2002; Pym, 2003) produced substantial levels of IFN-γ upon in vitro stimulation with ESAT-6:1-20 peptide or r-CyaA-ESAT-6 construct (FIG. 10). It is noteworthy that these IFN-γ levels produced were comparable to those produced following stimulation with purified protein derivative (PPD) of M. tuberculosis. The specificity of this T-cell responses was established by the observations that: (i) stimulation of these cells with unrelated Mal-E:40-54 peptide or r-CyaA-OVA:257-264 negative controls did not induce release of IFN-γ and (ii) splenocytes of mice infected with a control BCG (BCG::pYUB412) did not produce detectable IFN-γ after in vitro stimulation with ESAT-6:1-20 or r-CyaA-ESAT-6 (FIG. 10).

Mice, Infection, Immunization. Female, specific pathogen-free BALB/c (H-2^{d}) or C57BL/6 (H-2^{b}) mice (Iffa Crédo, L'Arbresle, France) were used at 6-12 weeks of age. Mice were infected (s.c. or i.v.) with 1×10⁶ or 1×10⁷ CFU/mouse of BCG::RD1 and were maintained in isolators in ABL-3 biohazard conditions in Pasteur Institute's animal facilities. T-cell responses were studied 3-4 weeks post-infection. Mouse immunization with r-CyaA was performed by one or two i.v. injections with 10 or 50 μg of appropriate r-CyaA in PBS. T-cell responses were studied 10-12 weeks post-immunization.

T-cell proliferation and cytokine production assays. Single-cell suspensions of spleen or lymph node cells were plated (1×10⁶ cell/well) onto 96-well flat-bottom plates in synthetic HL-1 medium (BioWhittaker, Walkersville, Md.) complemented with 2 mM L-glutamine, 100 IU penicillin/ml and 100 μg streptomycin/ml in the presence of various concentrations of synthetic peptides (Neosystems, Strasbourg, France) or 1-10 μg/ml of r-CyaA. For lymphoproliferation assays, cultures were pulsed with 1 μCi [methyl-³H]-thymidine (ICN, Orsay, France) for 16 h and cells were harvested for cpm counting.

For cytokine assays, culture supernatants were collected at 48 h for IL-2 detection and at 72 h for the other cytokines. IL-2 was quantified using a standard CTLL-2 bioassay. IL-4, IL-5 and IFN-γ were quantified by a sandwich ELISA using, respectively, BVD4-1D1, TRFK5 and R4-6A2 as capture monoclonal antibodies and biotin-conjugated BVD6-24G2, TRFK4 and XMG1.2 monoclonal antibodies (BD PharMingen, San Diego, Calif.). Standard curves were obtained with recombinant murine cytokines (BD PharMingen).

### Example 13

### Materials and Methods for Demonstrating the Scope of the Use of the Invention

Mice. Female C57BL/6 (H-2^{b}) mice from Iffa Credo (L'Arbresle, France) were used between 6 and 10 weeks of age. Female TAP1 knockout mice (Van Kaer, L., et al., 1992) onto a C57BL/6 background were a gift from A. Bandeira (Institut Pasteur, Paris, France) and were bred in our animal facilities.

Peptides and proteins. The synthetic peptides SIINFEKL and NGKLIAYPIAVEALS, corresponding respectively to the CD8⁺ T cell epitope encompassing the ovalbumin residues 257-264 (Bevan, M. J., 1976) and to the CD4⁺ T cell epitope corresponding to E. coli MalE protein residues 100-114 (REF) were purchased from Neosystem (Strasbourg, France). MalE protein was kindly given by J. M. Clément (Institut Pasteur) and ovalbumin was purchased from Sigma (Saint-Quentin Fallavier, France). Both were dissolved in PBS at 1 mg/ml.

Construction, production and purification of recombinant CyaA toxins with inserted CD4⁺ MalE and CD8⁺ OVA epitopes. To construct the hybrid cyaA alleles encoding the CyaA proteins carrying simultaneously the MalE and the OVA epitopes, appropriate unique restriction sites along the cyaA alleles were used for recombination of cyaA alleles encoded on a set of pT7CACT1-derived plasmids and carrying oligonucleotide inserts encoding for either the CD4⁺ MalE epitope (Loucka, J., et al., 2002) or the CD8⁺ OVA epitope (Osicka, R., et al., 2000), respectively. The insertion and the orientation of both oligonucleotides in cyaA gene were verified by restriction analysis of plasmids, the length of the corresponding expressed CyaA proteins was verified by 7.5% SDS-PAGE. The recombinant CyaA used in this study bear the NGKLIAYPIAVEALS sequence between amino acids 108 and 109 (CyaA-MalE), the SIINFEKL sequence between amino acids 336 and 337 (CyaA-OVA), or both sequences in their respective insertion site (CyaA-MalE-OVA). All constructs were genetically detoxified by insertion of a dipeptide sequence between residues 188 and 189.

The E. coli XL-1 Blue strain (Stratagene) was transformed with the constructed plasmids derived from pT7CACT1 and containing the accessory gene cyaC required for post-translational acylation of ACT (Osicka, R., et al., 2000). The cells were grown as described previously (Osicka, R., et al., 2000) and the expression of recombinant proteins was induced by adding of 1 mM IPTG. The CyaA proteins were extracted with 8M urea (Sebo, P., et al., 1991) and purified by DEAE-Sepharose and Phenyl-Sepharose chromatographies (Karimova, G., et al., 1998). The homogeneity of purified toxins was verified by 7.5% SDS-PAGE. Purified recombinant CyaA proteins concentrations were determined by the Bradford method.

CyaA E5, a genetically detoxified CyaA without insert, was kindly provided by D. Ladant (Institut Pasteur) and was used as a negative control.

Culture medium. Complete medium (CM) consisted of RPMI 1640 containing L-Alanyl-L-Glutamine dipeptide supplemented with 10% fetal calf serum (Valbiotech, Paris, France), 5×10⁻⁵ M of 2-ME and antibiotics (penicillin 100 U/ml, streptomycin 100 μg/ml).

Cell lines. The H-2^{b} restricted hybridoma CRMC3, specific for the 100-114 sequence of the MalE protein from E. coli was generated in our laboratory as previously described (Lo-Man, R., et al., 2000) and was maintained in CM. B3Z (Karttunen, J., et al., 1992), the CD8⁺T cell hybridoma specific for the K^{b} restricted OVA 257-264 peptide was a generous gift from N. Shastri (University of California, Berkeley, Calif.), and was maintained by adding 1 mg/ml of G418 and 400 μg/ml of hygromycin B to the CM. The EL-4 thymoma was obtained from American Type Culture Collection (Manassas, Va.) and maintained in CM.

BMDC generation. BMDCs were generated from bone marrow precursors as previously described (Inaba, K., et al., 1992). Briefly, bone marrow cells from C57BL/6 or TAP1 knockout mice were harvested, washed, and plated at 2.10⁵ cells/ml in CM with 1% of a GMCSF-containing supernatant. After 3 days of culture at 37° C., 7% CO₂, medium was added in the plates. The non-adherent and semi-adherent cells were recovered at day 7 or 8 by flushing the plates with PBS EDTA (5 mM) and washed before use. The recovered cells usually contained 60 to 70% of CD11c positive cells that all expressed CD11b. These BMDCs were CD40^{lo} and CD86^{lo}.

Antigen presentation assays. The stimulation of CRMC3 or B3Z T-cell hybridoma (10⁵ cells/well) was monitored by IL-2 release in the supernatants of 18-h cell cultures in the presence of BMDCs (10⁵ cells/well) in 96-well culture plates. In most experiments, BMDCs were pulsed for 4 to 5 hours with proteins or peptides at various concentrations (see legends of the figures) and washed three times before adding 10⁵ T cell hybridoma in 0.2 ml of CM. In the drug inhibition assay, the BMDCs were fixed with 0.05% glutaraldehyde (Sigma) after being pulsed and washed, and then the hybridoma were added. After 18 hours, culture supernatants were frozen for at least 2 hours at −80° C. Then, 10⁴ cells/well of the IL-2 dependent CTL-L cell line were cultured with 100 μl of these supernatants. After 48 hours, [³⁻H]-thymidine (50 μCi/ml, ICN, Orsay, France) was added to the wells and the cells were harvested 6 hours later with an automated cell harvester (Skatron, Lier, Norway). Incorporated thymidine was detected by scintillation counting. In all experiments, each point was done in duplicate.

Inhibitors and antibodies. Cycloheximide (CHX, used at 5 μg/ml), brefeldin A (BFA, 5 μg/ml), cytochalasin B (CCB, 5 μg/ml), leupeptin (50 μg/ml), pepstatin (50 μg/ml), chloroquine (50 and 150 μM), N-acetyl-L-leucinal-L-norleucinal (LLnL, 12 μg/ml) and N-acetyl-L-leucinal-L-methioninal (LLmL, 12 μg/ml), were all from Sigma-Aldrich (Saint-Louis, Mo.) and were dissolved in appropriate solvent according to manufacturer's advises. Lactacystin (Biomol, research Labs., Inc., Plymouth Meeting Pa.) was dissolved in water at 1 mg/ml and used at 10 μM final. The purified mAbs specific for murine CD11b (M1/70, rat IgG2b,K) and the corresponding isotype control were purchased from Pharmingen (Le Pont de Claix, France) and were used at 10 μg/ml.

Inhibition studies. For inhibition studies, BMDCs were first incubated with the drugs or antibodies for one hour in 0.1 ml of CM at 37° C., 7% CO₂. Then, Ags were added in 0.1 ml of CM at the final concentrations indicated in the legends of the figures, in the continuous presence of the inhibitors. In the assays using anti-CD11b or isotype control antibodies, the cells were washed three times after 5 hours of incubation with both Ags and antibodies, and 10⁵ T cell hybridomas were added. In the assays using drugs, the cells were washed after the 5-hours incubation and fixed using glutaraldehyde 0.05% for 2 min at 37° C. (Sigma) and lysine 0.2 M (Sigma). After washing three times, the T cell hybridoma were added to the wells in 0.2 ml CM.

For inhibition of clathrin-mediated endocytosis by K⁺ depletion following hypotonic shock, DC (10⁵/well) were incubated for 30 min in serum-free synthetic OptiMEM medium (Life Technologies) supplemented with 5.10⁻⁵ M 2-ME, 100 U/ml penicillin and 100 μg/ml streptomycin. DCs were then incubated for 5 min in hypotonic medium (OptiMEM medium and ultrapure H₂O, 50/50) and finally for 30 min in K⁺-free (140 mM NaCl, 20 mM HEPES-NaOH, 1 mM CaCl₂, 1 mM MgCl₂, 1 mg/ml glucose and 0.5% BSA) or K⁺-containing (10 mM KCl, 130 mM NaCl, 20 mM HEPES-NaOH, 1 mM CaCl₂, 1 mM MgCl₂, and 0.5% BSA). Ags were added to the wells at the concentrations indicated in the figure legends and 1 hour later, DCs were washed in PBS and CM was added for 4 hours to allow Ag processing. DCs were washed and fixed as described previously and T cell hybridomas were added to the wells for 18 hours.

Mouse immunization. C57BL/6 mice were i.v. injected with 50 μg of CyaA-OVA, CyaA-MalE, CyaA-MalE-OVA or CyaA E5 diluted in 0.1 ml of PBS.

In vitro cytotoxicity assays. Splenocytes from immunized mice were isolated 7 days after CyaA injection and in vitro restimulated for 5 days with OVA₂₅₇₋₂₆₄ peptide (1 μg/ml) in the presence of syngeneic irradiated naive spleen cells. The cytotoxic activity was determined in a 5-hour in vitro [⁵¹Cr]-release assay as previously described (Fayolle, C., et al., 1996). Briefly, EL4 (H-2^{b}) tumor cells loaded with 50 μM of the OVA₂₅₇₋₂₆₄ peptide were used as target cells for H-2^{b} effector cells. Various effector to target ratios were used and all assays were done in duplicate. In each assay, EL-4 cells incubated in the absence of the peptide were used as control for nonspecific lysis. [⁵¹Cr]-release in each well was counted using a MicroBeta Trilux liquid scintillation Counter (Wallac, Turku, Finland). Percentage of specific lysis was calculated as 100×(experimental release−spontaneous release)/(maximal release−spontaneous release). Maximum release was obtained by adding 10% Triton X405 to target cells and spontaneous release was determined with target cells incubated in CM.

Cytokine ELISA assay. Splenocytes from immunized mice were restimulated in vitro in the presence or absence of 1 μg/ml of MalE₁₀₀₋₁₁₄ or OVA₂₅₇₋₂₆₄ peptides and the culture supernatants were harvested after 72 hours. IL-4, IL-5, and IFN-δ concentrations were then measured in these supernatants by a standard sandwich ELISA. Maxisorp plates (Nunc, Roskilde, Denmark) were coated with unconjugated anti-IL-4, anti-IL-5, or anti-IFN-δ capture antibodies (BVD4-1D11, TRFK5, R4-6A2 clones respectively, Pharmingen) and detection was done using corresponding biotinylated mAb (BVD6-24G2, TRFK4, XMG1.2 clones, Pharmingen). The plates were developed using streptavidin-HRP (Pharmingen) and o-Phenylenediamine (Sigma-Aldrich) as substrate. All dosages were performed in duplicate. The assays were standardized with recombinant murine cytokines (Pharmingen) and results are expressed in pg/ml.

### Example 14

### CyaA-MalE is More Efficient than the MalE Protein in CD4⁺ T Cell Epitope Delivery into MHC Class II Presentation Pathway

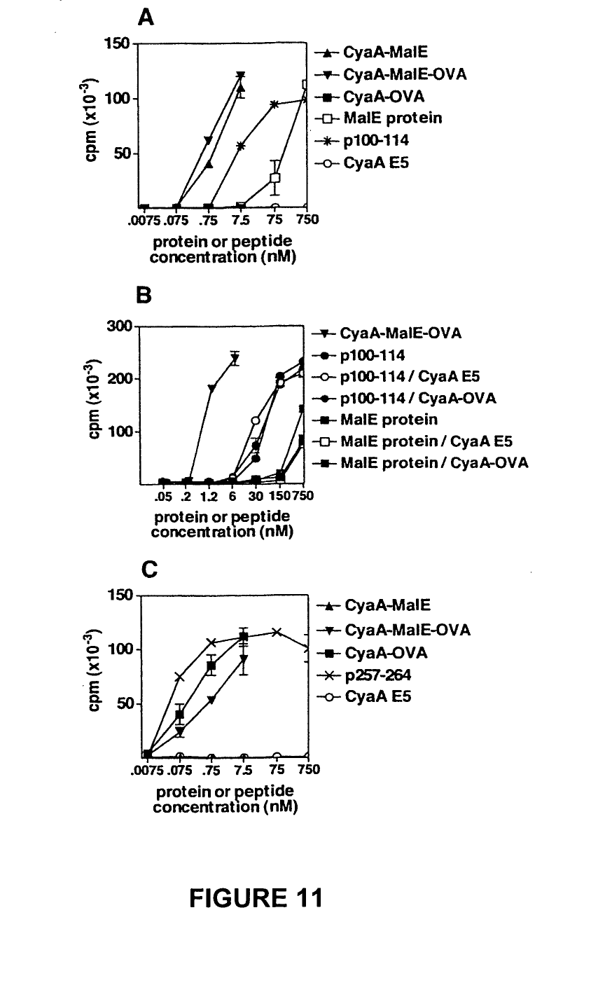

Using a MalE CD4⁺ T cell epitope as reporter, it has been previously shown that recombinant CyaA-MalE delivers the NGKLIAYPIAVEALS MalE₁₀₀₋₁₁₄ peptide into MHC class II presentation pathway of splenocytes (Loucka, J., et al., 2002). The efficiency of this delivery as compared to MalE protein was evaluated next. Using BMDCs, which are CD11b positive (data not shown), the presentation of CyaA carrying the MalE NGKLIAYPIAVEALS CD4⁺ T cell epitope at position 108 was compared to MalE protein. APCs were incubated with serial dilutions of each protein and I-A^{b}-NGKLIAYPIAVEALS complexes apparition at their surface was monitored with CRMC3, a CD4⁺ T cell hybridoma specific for this MHC-peptide complex (Lo-Man, R., et al., 2000). As expected (FIG. 11A), BMDCs incubated with CyaA-MalE efficiently stimulated IL-2 secretion by CRMC3. Moreover, 100 times higher concentration of MalE protein were required to reach the same level of T cell hybridoma stimulation as with CyaA-MalE. As previously shown with splenocytes (Loucka, J., et al., 2002), BMDCs incubated with CyaA-MalE were also 10 fold more efficient than BMDCs loaded with the free MalE₁₀₀₋₁₁₄ peptide in stimulating CRMC3.

To exclude that this potentiation was due to a non-specific stimulatory effect of the CyaA or of some component in the CyaA preparation, BMDCs were incubated with a constant concentration of CyaA E5 or CyaA-OVA and various concentrations of MalE₁₁₀₋₁₁₄ peptide or MalE protein. The efficiency of I-A^{b}-NGKLIAYPIAVEALS complexes presentation to CRMC3 was then monitored. As shown in FIG. 1B, no potentiation of MalE₁₀₀₋₁₁₄ peptide or MalE protein presentation was observed in the presence of CyaA E5 or CyaA-OVA. These results confirm that the potentiation of MHC class II-restricted presentation of CD4⁺ T cell epitope delivered by CyaA is not due to BMDC activation by CyaA or contaminant.

### Example 15

### CyaA-MalE-OVA Simultaneously Delivers Model CD4⁺ and CD8⁺ T Cell Epitopes for MHC I and II Presentation

In a previous report, it was shown that CyaA carrying three different CD8⁺ T cell epitopes simultaneously induces in vivo protective CTL responses against these epitopes (Fayolle, C., et al., 2001). Therefore, it was determined whether CyaA could deliver both CD4⁺ and CD8⁺ T cell epitopes to BMDCs for Ag presentation to specific T cell hybridoma. Therefore, CyaA-MalE-OVA, a recombinant CyaA bearing both MalE (class II-restricted) and OVA (class I-restricted) epitopes was compared to CyaA-MalE and CyaA-OVA in a presentation assay. To make this comparison possible, the MalE CD4⁺ T cell epitope was inserted between amino acids 108 and 109 of CyaA-MalE and CyaA-MalE-OVA. The OVA CD8⁺ T cell epitope was inserted between amino acids 336 and 337 in CyaA-OVA and CyaA-MalE-OVA. To detect the presence of K^{b}-SIINFEKL complexes on BMDCs B3Z, a CD8⁺ T cell hybridoma specific for the OVA₂₅₇₋₂₆₇ peptide (Karttunen, J., et al., 2002) was used. As shown in FIGS. 11A and 11C, BMDCs incubated with CyaA-MalE-OVA stimulated both CRMC3 and B3Z T cell hybridoma. Moreover, CyaA-MalE-OVA was as efficient as CyaA-MalE in MalE₁₀₀₋₁₁₄ peptide delivery into MHC class II presentation pathway. An equivalent K^{b}-SIINFEKL complexes presentation to B3Z following incubation of CyaA-MalE-OVA or CyaA-OVA with BMDCs was also observed. These results confirm that CyaA simultaneously delivers epitopes inserted in its AC domain to MHC I and II molecules and that the efficiency of delivery of one epitope is not affected by the insertion of another epitope into CyaA's AC domain. Particularly, the potentiation of MHC class II presentation was still observed with the CyaA bearing both OVA and MalE epitopes.

### Example 16

### The Interaction of CyaA with CD11b on BMDCs is Required for the Potentiation of Delivery of the Reporter CD4⁺ T Cell Epitope

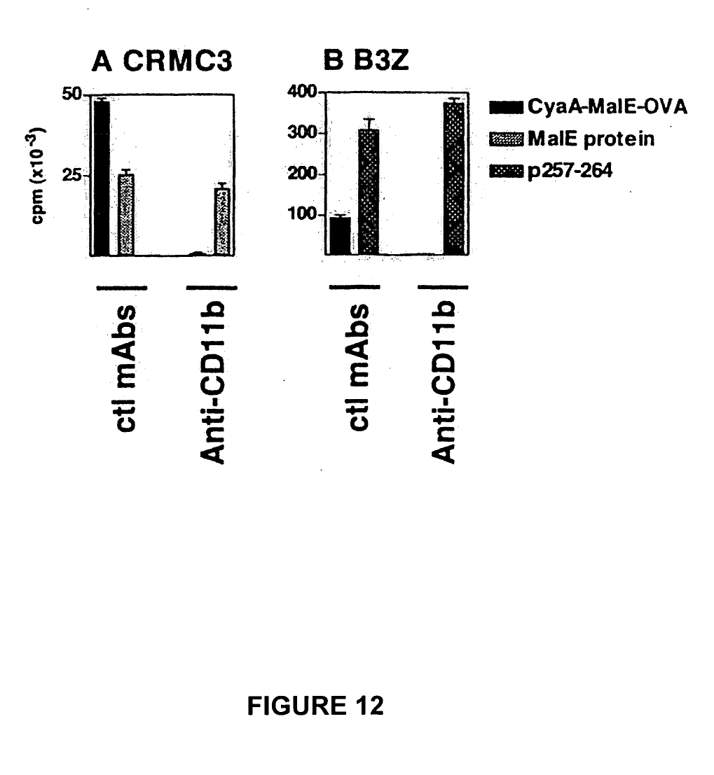

The potentiation of MHC class II-restricted presentation on CyaA delivery could be explained by the specific interaction of this protein with its CD11b receptor (Guermonprez, P., et al., 2001), which is expressed on BMDCs. To test this hypothesis, BMDCs were first incubated either with 10 μg/ml anti-CD11b mAbs or with the same concentration of isotype control mAbs. As shown in FIG. 2A, pre-incubation of the APCs with anti-CD11b mAbs totally and specifically abrogated the presentation of MalE₁₀₀₋₁₁₄ peptide to CRMC3 following CyaA-MalE-OVA delivery. As expected, the pre-incubation of BMDCs with the mAbs did not affect the presentation of the MalE protein to the hybridoma. It was also confirmed that BMDCs incubation with anti-CD11b prevents the generation of K^{b}-SIINFEKL complexes from CyaA-OVA-MalE (FIG. 12B) without affecting the free OVA2-264 peptide presentation to B3Z. These results show that the high efficiency of CyaA to be delivered to both MHC class I and class II pathway is dependent on CyaA-CD11b specific interaction.

### Example 17

### MalE Peptide Delivery into MHC Class II Presentation Pathway by CyaA-MalE OVA Does Not Require Proteasome Activity Nor TAP Transporters

Previous studies have demonstrated that CyaA interaction with CD11b results in direct AC domain translocation into target cell cytosol. The subsequent processing of this domain to generate peptides for MHC class I-restricted presentation requires proteasome and is dependent on TAP transporters (Guermonprez, P., et l., 1999). It has been reported that some endogenous Ags are processed in the cytosol for MHC class II presentation by an alternative pathway that requires the proteasome and calpain (Lich, J. D., et al., 2000). The peptides released in the cytosol are then transported into endocytic compartments along a poorly understood mechanism. Therefore, the proteasome requirement of CyaA-MalE-OVA for MHC class II-restricted MalE₁₀₀₋₁₁₄ peptide presentation to CRMC3 was tested. BMDCs were incubated for one hour with lactacystin, a 20S proteasome inhibitor (Fenteany, G., et al., 1995; Craiu, A., et al., 1997), and the Ags were then added.

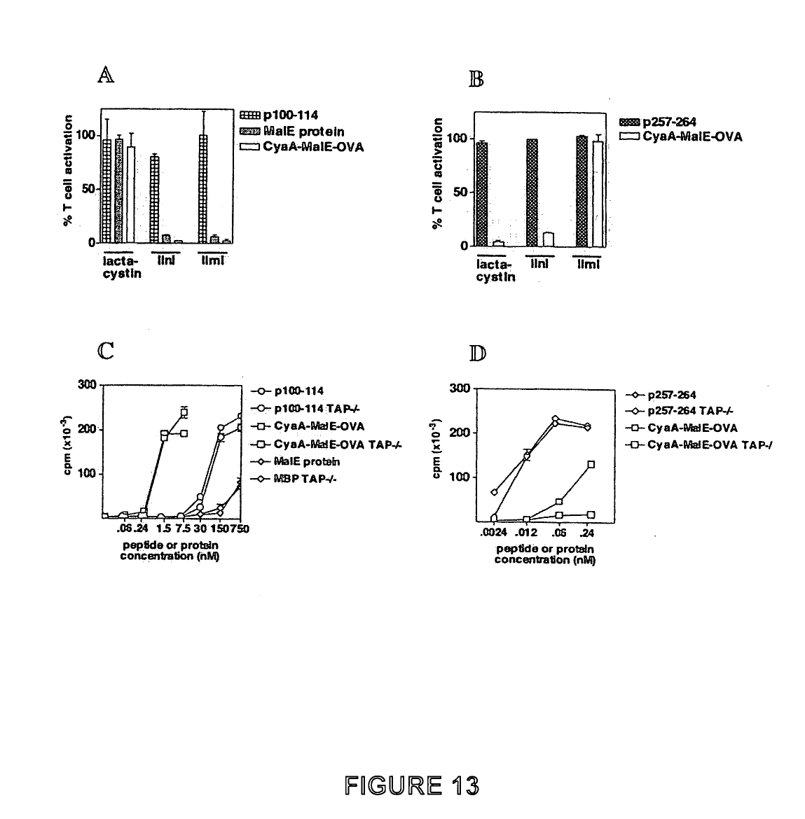

As shown in FIG. 13A, the inhibition of proteasome activity did not abrogate I-A^{b}-MalE₁₁₀₋₁₁₄ complexes formation and presentation to CRMC3. As expected, the free peptide and MalE protein were still presented by the BMDCs treated with lactacystin. In contrast, OVA₂₅₇₋₂₆₄ peptide presentation by CyaA-MalE-OVA delivery was totally abrogated by lactacystin (FIG. 13B).

The effect of LLnL (a cathepsin and proteasome inhibitor) and LLmL (a cathepsin inhibitor) (Rock, K. L., et al., 1997) on MalE₁₀₀₋₁₁₄ peptide presentation to CRMC3 by BMDCs was then compared. As shown in FIG. 13A, both inhibitors prevented MalE₁₀₀₋₁₁₄ peptide presentation upon CyaA-MalE-OVA delivery. This demonstrated the requirement for cathepsin L or B in this processing pathway. As a control, LLnL but not LLmL prevented OVA peptide presentation to B3Z following CyaA-MalE-OVA delivery. Thus, after its entry into BMDC, CyaA AC domain is processed to generate peptides for MHC class II-restricted presentation by a mechanism that does not require proteasome activity, but depends upon cathepsin L or B, two cysteine proteases of the endocytic pathway.

To further confirm that the processing of class I and class II epitopes from the AC domain follows distinct pathways after AC domain delivery to APCs, the TAP requirement for CyaA presentation by MHC class II molecules was tested. BMDCs were generated from TAP1 knockout mice and used in a presentation assay to CRMC3. As shown in FIG. 13C, CyaA-MalE-OVA efficiently delivered MalE₁₀₀₋₁₁₄ peptide into the MHC class II presentation pathway of both WT and TAP1 knock out BMDCs (Van Kaer, L., et al., 1992). As expected, MalE₁₀₀₋₁₁₄ peptide delivery to MHC class II molecules was also not dependent on TAP transporters when MalE protein or MalE₁₀₀₋₁₁₄ peptide were used as Ags. As previously published (Guermonprez, P., et al., 2002), CyaA-MalE-OVA delivery into BMDCs MHC class I presentation pathway was shown to require the presence of TAP1 transporters in the APCs (FIG. 13D). These results further confirm that generation of MHC class I and class II peptides from CyaA AC domain follows two distinct pathways. Moreover, the requirement of cathepsin activity for MalE₁₀₀₋₁₁₄ peptide presentation on CyaA-MalE-OVA delivery suggests that the endocytic route of processing might be responsible for CyaA degradation and entry into MHC class II presentation pathway.

### Example 18

### CyaA-MalE-OVA Processing for MHC II Presentation Requires Endosomal Proteases and Vacuolar Acidification

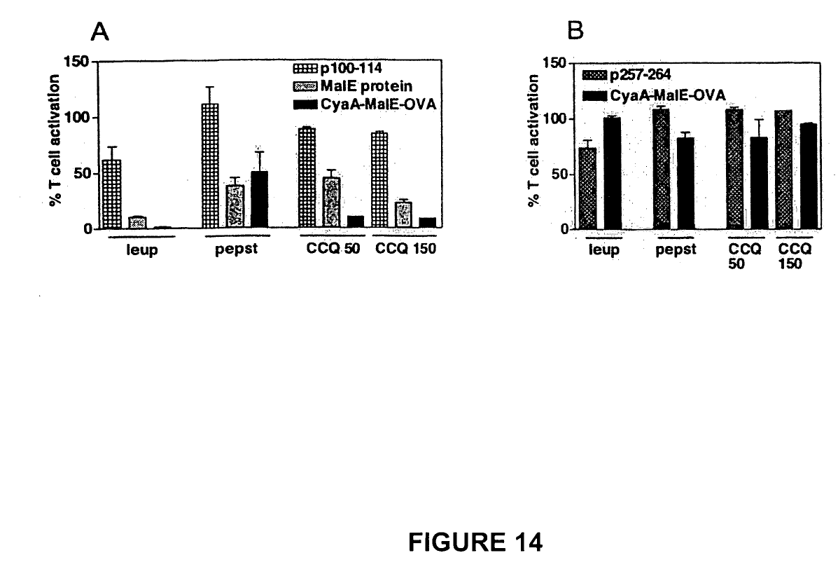

After internalization of exogenous soluble Ag, peptide ligands for MHC II presentation are generated in endosomes and lysosomes by proteolysis of the proteins by a set of proteases that are sequentially activated (Villadangos, J. 2001). As cathepsin activity is required to generate MalE₁₀₀₋₁₁₄ peptide presentation after CyaA-MalE-OVA delivery, whether others endocytic proteases are required for I-A^{b}-MalE₁₀₀₋₁₁₄ complexes formation was tested. Leupeptin (Umezawa, H. 1976), an inhibitor for serine and cysteine proteases totally blocked MalE₁₀₀₋₁₁₄ peptide presentation to CRMC3 when CyaA-MalE-OVA was used as Ag, but did not affect the presentation of the free peptide (FIG. 14A). It should be mentioned that serine proteases may indeed be required to generate N-terminal end of the MalE epitope. Pepstatin (Umezawa, H. 1976; Mizuochi, T., et al., 1994), an inhibitor for aspartate proteases also partially inhibited MalE100-114 peptide presentation after CyaA-MalE-OVA delivery. Free MalE₁₀₀₋₁₁₄ peptide presentation remained unaffected in the presence of the drug. These results show that endocytic proteases are involved in AC domain degradation for MHC class II peptide generation. Therefore, these results indicate that the CyaA AC domain reaches the classical endocytic route to be processed by proteases.

Vacuolar acidification is an important factor, which controls the sequential activation of endocytic proteases. Therefore, chloroquine, an inhibitor of endocytic vesicle acidification was used to confirm that MalE₁₀₀₋₁₁₄ peptide presentation on CyaA delivery occurs after endocytic processing. As shown in FIG. 14A, chloroquine strongly diminishes the presentation of MalE₁₀₀₋₁₁₄ peptide following CyaA-MalE-OVA and MalE protein delivery whereas free peptide presentation remains unaffected. However, it is confirmed that OVA₂₅₇₋₂₆₄ peptide presentation is not dependent on vacuolar acidification when CyaA-MalE-OVA is used as Ag (Guermonprez, P., et al., 2000a, b). These results demonstrate that AC domain processing into BMDCs to generate MHC class II restricted peptides is dependent on vacuolar acidification and endocytic proteases. They also suggest that CyaA AC domain can be simultaneously translocated into BMDCs cytosol to be further processed by proteasome and captured in vesicles that follow the endocytic route of processing. Alternatively, it could is possible that after binding to CD11b, CyaA is endocytosed and then, translocated to cytosol.

### Example 19

### MalE Epitope Delivery by CyaA-MalE-OVA is Sensitive to Golgi Disruption by BrefeldinA and Protein Synthesis Inhibition by Cycloheximide

Presentation of MHC II-peptide complexes at APC surface requires the degradation of exogenous Ag but also the association of the generated peptides with MHC class II molecules (Gordon, S. V., et al., 1999). In the classical endocytic pathway, newly synthesized MHC class II molecules are required. These molecules leave the ER through the Golgi and reach the trans-golgi network (TGN) where they are sent towards the endocytic pathway.

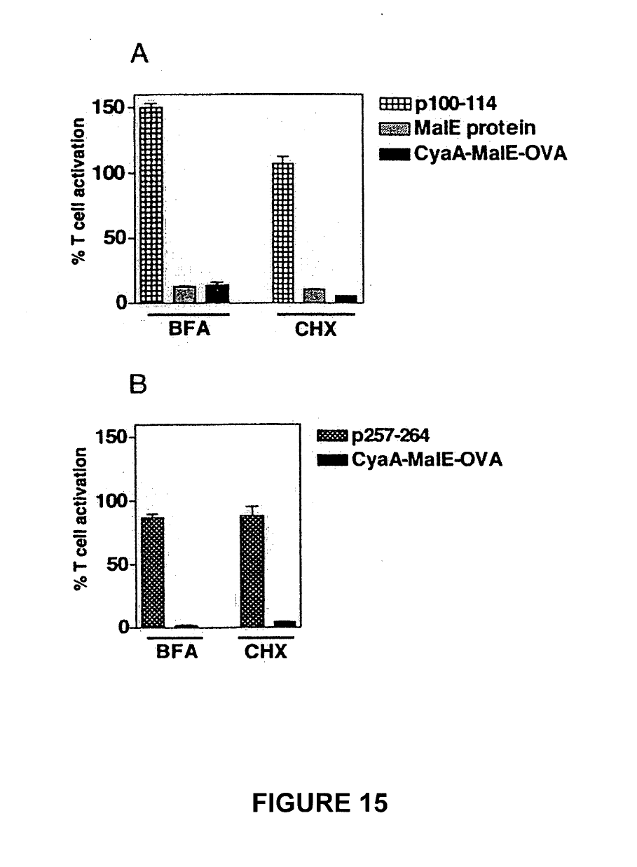

To determine whether I-A^{b}-MalE₁₀₀₋₁₁₄ peptide complexes generation after CyaA-MalE-OVA delivery requires nascent MHC class II molecules, cycloheximide (CHX), an inhibitor of protein synthesis was used. As shown in FIG. 15A, BMDCs that have been pre-incubated with CHX before addition of CyaA-MalE-OVA or MalE protein did not stimulate IL-2 secretion by CRMC3. As expected, CHX did not inhibit the presentation of the free peptide to T cell hybridoma. Moreover, OVA₂₅₇₋₂₆₄ peptide presentation to B3Z following CyaA-MalE-OVA delivery was also totally abrogated by CHX (FIG. 15B). Thus, newly synthesized proteins are necessary for MHC class II-restricted presentation of the MalE reporter T cell epitope inserted into CyaA AC domain.

To determine whether MHC class II molecules that present MalE₁₀₀₋₁₁₄ peptide reach early and late endosomes towards Golgi, Brefeldin A (BFA), an inhibitor of Golgi transport (Doms, R. W., et al., 1989; Pelham, H. R. 1991) was used. Here again, the presentation of MalE₁₀₀₋₁₁₄ peptide after its delivery to BMDCs by CyaA-MalE-OVA or MalE protein was totally abrogated when the APCs had been treated with BFA (FIG. 15A). The presentation of the free peptide was not affected. From these experiments it is clear that neosynthesis of MHC class II molecules is necessary for BMDCs to present the MalE epitope delivered by CyaA-MalE-OVA and that trafficking through Golgi towards TGN allows these newly synthesized molecules to reach the endocytic pathway.

### Example 20

### MalE Epitope Delivery by CyaA-MalE-OVA Does Not Depend on Actin Filament Polymerization but Requires Clathrin-Coated Pits

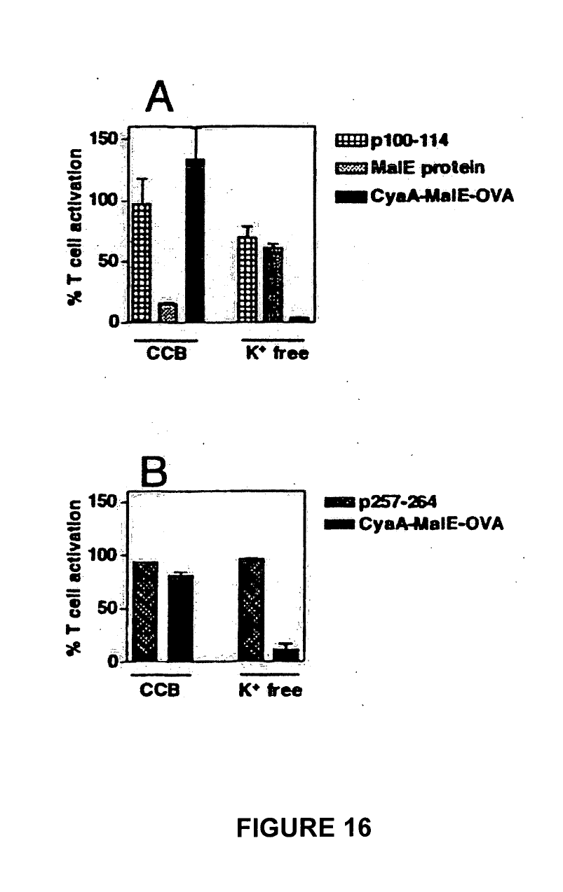

The internalization of CyaA and the subsequent MHC class I-restricted presentation of the OVA peptide inserted in its AC domain have already been shown to be independent on phagocytosis (Guermonprez, P., et al., 2000a, b). However, it can not be excluded that some molecules of CyaA translocate their AC domain into APCs cytoplasm whether others are captured and processed as classical exogenous Ag to give rise to MHC class II-restricted peptides. It was first tested whether actin-dependent capture was implicated in MalE epitope delivery for efficient MHC class II-restricted presentation. In this experiment, cytochalasin B (CCB), a drug that prevents actin filament polymerization and impairs macropinocytosis, phagocytosis, and also caveolae-mediated endocytosis (Gottlieb, T. A., et al., 1993) was used. As shown in FIG. 16, CCB did not inhibit either MalE nor OVA₂₅₇₋₂₆₄ peptide presentation to their respective specific T cell hybridoma following CyaA-MalE-OVA delivery. As expected, the presentation of the MalE protein was totally abrogated by the inhibitor whereas the free MalE₁₀₀₋₁₁₄ peptide was still presented to CRMC3. These results show that AC domain delivery to MHC class I and II molecules does not require CyaA phagocytosis, macropinocytosis, or caveolae-mediated endocytosis by BMDCs.

As CyaA interacts with CD11b on APC cell surface it was tested, whether CyaA was endocytosed by a clathrin-dependent process. K⁺ depletion following hypotonic shock (Larkin, J. M., et al., 1983; Madshus, I. H., et al., 1987; Bayer, N., et al., 2001) was used to test if clathrin coated pits were required for MHC class I and class II presentation of CyaA-MalE-OVA. K⁺ depletion following hypotonic shock was performed by BMDCs exposure to hypotonic medium followed by incubation in the absence of extracellular potassium. This treatment results in dissociation of clathrin coats from the plasma membrane and nonproductive assembly of clathrin cages in the cytoplasm. Internalization of membrane proteins that interact with AP2 clathrin adapter complex through cytoplasmic amino acid sequences is therefore impaired. As shown in FIG. 16, both MalE₁₀₀₋₁₁₄ and OVA₂₅₇₋₂₆₄ peptide presentation to their respective T cell hybridoma after CyaA-MalE-OVA delivery was totally abrogated by K⁺ depletion, whereas MalE protein or free peptides presentation was not inhibited.

These results demonstrate that CyaA-MalE-OVA clathrin-mediated endocytosis is required for both class I and class II restricted presentation. This was surprising, as it was believed that CyaA directly translocated its AC domain into cytosol from plasma cell membrane, without being endocytosed. Instead, these results suggest that CyaA AC domain is translocated from clathrin-coated vesicles after its endocytosis.

### Example 21

### CyaA-MalE-OVA Induces OVA-Specific CD8⁺ T Cell Responses and MalE-Specific CD4^{′} T Cell Responses in Vivo

The great efficiency of CyaA to induce CTL responses against different CD8⁺ T cell epitopes (Fayolle, C., et al. 2001), and proliferative responses against MalE CD4⁺ T cell epitope (Loucka, J., et al., 2002) has previously been demonstrated. After in vitro studies demonstrating that CyaA is a potent vehicle to deliver both CD4⁺ and CD8⁺ T cell epitopes to BMDCs for Ag presentation, the efficiency of CyaA-MalE-OVA in the simultaneous in vivo delivery of these epitopes was tested. Mice were immunized with 50 μg of CyaA-MalE, CyaA-OVA, CyaA-MalE-OVA or CyaA E5 by i.v. route, without adjuvant. The T cell responses were monitored seven days after injection.

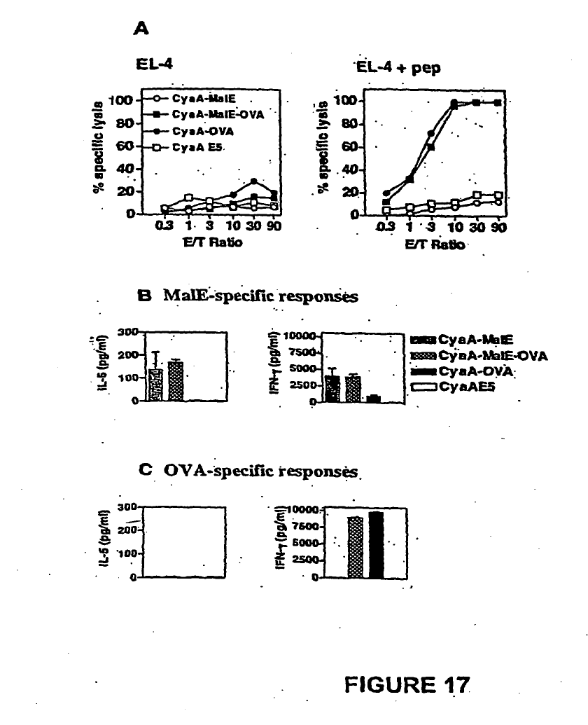

As a readout for CD8⁺ T cell responses, the cytotoxic activity of splenocytes from immunized mice against target cells loaded with the OVA₂₅₇₋₂₆₄ peptide was tested. As shown in FIG. 17A, both CyaA-MalE-OVA and CyaA-OVA induced specific CTL responses against the OVA epitope. As expected, no response was detected when mice had received CyaA-MalE or CyaA E5. The cytokine secretion by CyaA-primed T cells was also analyzed. Splenocytes from immunized mice were restimulated with or without the corresponding peptide and IFN-γ and IL-5 specific secretions in 72 h culture supernatants were monitored by ELISA. As previously reported for a LCMV epitope (Dadaglio, G., et al., 2000), CyaA-OVA induced a Th1-like polarized OVA-specific T cell response, characterized by a strong IFN-γ production, but no IL-5 secretion (FIG. 17C). No IL-4 and IL-10 were detected in these culture supernatants. These results show that CyaA-MalE-OVA is as immunogenic as CyaA-OVA for in vivo induction of Th1-polarized CD8⁺ T cell responses.

The CD4⁺ T cell responses induced by CyaA-MalE-OVA as compared to CyaA-OVA were also analyzed. As readout, the cytokine secretion of splenocytes in vitro restimulated with the MalE₁₀₀₋₁₁₄ peptide was monitored. As shown in FIG. 17B, both CyaA-MalE-OVA and CyaA-MalE induced a specific IFN-γ secretion by immune splenocytes. IL-5 secretion was also detected in three experiments out of four, but the levels remained very low (see FIG. 17B) showing that the CD4⁺ T cell responses induced by CyaA are mainly Th1-polarized as the CD8⁺ T cell responses. Here again, no IL-10 or IL-4 were detectable in the supernatants.

These results further confirm the capacity of CyaA simultaneously to deliver both class I and class II epitopes for in vivo T cell priming. Moreover, the efficiency of such simultaneous delivery is similar to the single epitope delivery, and both CD4⁺ and CD8⁺ T cell responses appear to be Th1 polarized.

Furthermore, the ESAT-6 (Rv3875, 95 amino acids) or CFP-10 (Rv3874, 100 amino acids) M. tuberculosis genomic sequences can be delivered by CyaA and CyaA affects the dose-response or detection frequency of M. tuberculosis specific IFN-γ producing cells, which enhances immunodiagnosis of TB.

### REFERENCES

The following references are cited herein. The entire disclosure of each reference is relied upon and incorporated by reference herein.

1. 2000. Control and prevention of tuberculosis in the United Kingdom: code of practice 2000. Joint Tuberculosis Committee of the British Thoracic Society. Thorax 55:887-901.

2. Arend, S. M., Andersen, P., van Meijgaarden, K. E., Skjot, R. L., Subronto, Y. W., van Dissel, J. T., and Ottenhoff, T. H. 2000. Detection of active tuberculosis infection by T cell responses to early-secreted antigenic target 6-kDa protein and culture filtrate protein 10. J Infect Dis 181:1850-1854.

3. Arend, S. M., van Meijgaarden, K. E., de Boer, K., de Palou, E. C., van Soolingen, D., Ottenhoff, T. H., and van Dissel, J. T. 2002. Tuberculin skin testing and in vitro T cell responses to ESAT-6 and culture filtrate protein 10 after infection with Mycobacterium marinum or M. kansasii. J Infect Dis 186:1797-1807.

4. Ballard, J. D., R. J. Collier, and M. N. Starnbach. 1996. Anthrax toxin-mediated delivery of a cytotoxic T-cell epitope in vivo. Proc Natl Acad Sci USA. 93:12531.

5. Bayer, N., D. Schober, M. Huttinger, D. Blaas, and R. Fuchs. 2001. Inhibition of clathrin-dependent endocytosis has multiple effects on human rhinovirus serotype 2 cell entry. J Biol Chem 276:3952.

6. Behr, M. A., Wilson, M. A., Gill, W. P., Salamon, H., Schoolnik, G. K., Rane, S., and Small, P. M. 1999. Comparative genomics of BCG vaccines by whole-genome DNA microarray. Science 284:1520-1523.

7. Berthet, F. X., Rasmussen, P. B., Rosenkrands, I., Andersen, P., and Gicquel, B. 1998. A Mycobacterium tuberculosis operon encoding ESAT-6 and a novel low-molecular-mass culture filtrate protein (CFP-10). Microbiology 144:3195-3203.

8. Bevan, M. J. 1976. Minor H antigens introduced on H-2 different stimulating cells cross-react at the cytotoxic T cell level during in vivo priming. J Immunol 117:2233.

9. Bona, C. A., S. Casares, and T. D. Brumeanu. 1998. Towards development of T-cell vaccines. Immunology Today. 19:126.

10. Boonstra, A., C. Asselin-Paturel, M. Gilliet, C. Crain, G. Trinchieri, Y. J. Liu, and A. O'Garra. 2003. Flexibility of Mouse Classical and Plasmacytoid-derived Dendritic Cells in Directing T Helper Type 1 and 2 Cell Development: Dependency on Antigen Dose and Differential Toll-like Receptor Ligation. J Exp Med. 197:1.

11. Brock, I., Munk, M. E., Kok-Jensen, A., and Andersen, P. 2001. Performance of whole blood IFN-gamma test for tuberculosis diagnosis based on PPD or the specific antigens ESAT-6 and CFP-10. Int J Tuberc Lung Dis 5:462-467.

12. Buddle, B. M., N. A. Parlane, et al. (1999). “Differentiation between Mycobacterium bovis BCG-vaccinated and M. bovis-infected cattle by using recombinant mycobacterial antigens.” Clin Diagn Lab Immunol 6(1): 1-5.

13. Cockle, P. J., Gordon, S. V., Lalvani, A., Buddle, B. M., Hewinson, R. G., and Vordermeier, H. M. 2002. Identification of novel Mycobacterium tuberculosis antigens with potential as diagnostic reagents or subunit vaccine candidates by comparative genomics. Infect Immun 70:6996-7003.

14. Craiu, A., M. Gaczynska, T. Akopian, C. F. Gramm, G. Fenteany, A. L. Goldberg, and K. L. Rock. 1997. Lactacystin and clasto-lactacystin beta-lactone modify multiple proteasome beta-subunits and inhibit intracellular protein degradation and major histocompatibility complex class I antigen presentation. J Biol Chem 272:13437.

15. Dadaglio, G., Moukrim, Z., Lo-Man, R., Sheshko, V., Sebo, P., and Leclerc, C. 2000. Induction of a polarized Th1 response by insertion of multiple copies of a viral T-cell epitope into adenylate cyclase of Bordetella pertussis. Infect Immun 68:3867-3872.

16. Doms, R. W., G. Russ, and J. W. Yewdell. 1989. Brefeldin A redistributes resident and itinerant Golgi proteins to the endoplasmic reticulum. J Cell Biol. 109:61.

17. El Azami El Idrissi, M., D. Ladant, and C. Leclerc. 2002. The adenylate cyclase of Bordetella pertussis: a vector to target antigen presenting cells. Toxicon. 40:1661.

18. Ewer, K., Deeks, J., Alvarez, L., Bryant, G., Waller, S., Andersen, P., Monk, P., and Lalvani, A. 2003. Comparison of T-cell-based assay with tuberculin skin test for diagnosis of Mycobacterium tuberculosis infection in a school tuberculosis outbreak. Lancet 361:1168-1173.

19. Fayolle, C., A. Osickova, R. Osicka, T. Henry, M. J. Rojas, M. F. Saron, P. Sebo, and C. Leclerc. 2001. Delivery of multiple epitopes by recombinant detoxified adenylate cyclase of Bordetella pertussis induces protective anti-viral immunity. J Virol 75:7330.

20. Fayolle, C., D. Ladant, G. Karimova, A. Ullmann, and C. Leclerc. 1999. Therapy of murine tumors with recombinant Bordetella pertussis adenylate cyclase carrying a cytotoxic T cell epitope. J Immunol. 162:4157.

21. Fayolle, C., P. Sebo, D. Ladant, A. Ullmann, and C. Leclerc. 1996. In vivo induction of CTL responses by recombinant adenylate cyclase of Bordetella pertussis carrying viral CD8+ T cell epitopes. J Immunol. 156:4697-4706.

22. Fenteany, G., R. F. Standaert, W. S. Lane, S. Choi, E. J. Corey, and S. L. Schreiber. 1995. Inhibition of proteasome activities and subunit-specific amino-terminal threonine modification by lactacystin. Science. 268:726.

23. Germain, R. N. 1994. MHC-dependent antigen processing and peptide presentation: providing ligands for T lymphocyte activation. Cell. 76:287.

24. Goletz, T. J., K. R. Klimpel, S. H. Leppla, J. M. Keith, and J. A. Berzofsky. 1997. Delivery of antigens to the MHC class I pathway using bacterial toxins. Human Immunol. 54:129.

25. Gordon, S. V., Brosch, R., Billault, A., Garnier, T., Eiglmeier, K., Cole, S. T. 1999. Identification of variable regions in the genomes of tubercle bacilli using bacterial artificial chromosome arrays. Mol Microbiol. 32: 643-655.

26. Gottlieb, T. A., I. E. Ivanov, M. Adesnik, and D. D. Sabatini. 1993. Actin microfilaments play a critical role in endocytosis at the apical but not the basolateral surface of polarized epithelial cells. J Cell Biol 120:695.

27. Guermonprez, P., C. Fayolle, G. Karimova, A. Ullmann, C. Leclerc, and D. Ladant. 2000. Bordetella pertussis adenylate cyclase toxin: a vehicle to deliver CD8-positive T-cell epitopes into antigen-presenting cells. Methods Enzymol. 326:527.

28. Guermonprez, P., Fayolle, C., Rojas, M. J., Rescigno, M., Ladant, D., and Leclerc, C. 2002. In vivo receptor-mediated delivery of a recombinant invasive bacterial toxoid to CD11c+CD8 alpha-CD11bhigh dendritic cells. Eur J Immunol 32:3071-3081.

29. Guermonprez, P., D. Ladant, et al. (1999). “Direct delivery of the Bordetella pertussis adenylate cyclase toxin to the MHC class I antigen presentation pathway.” J Immunol 162(4): 1910-6.

30. Guermonprez, P., D. Ladant, G. Karimova, A. Ullmann, and C. Leclerc. 1999. Direct delivery of the Bordetella pertussis adenylate cyclase toxin to the MHC class I antigen presentation pathway. J Immunol. 162:1910.

31. Guermonprez, P., Fayolle, C., Karimova, G., Ullmann, A., Leclerc, C., and Ladant, D. 2000. Bordetella pertussis adenylate cyclase toxin: a vehicle to deliver CD8-positive T-cell epitopes into antigen-presenting cells. Methods Enzymol 326:527-542.

32. Guermonprez, P., C. Fayolle, M. J. Rojas, M. Rescigno, D. Ladant, and C. Leclerc. 2002. In vivo receptor-mediated delivery of a recombinant invasive bacterial toxoid to CD11c+CD8 alpha-CD11bhigh dendritic cells. Eur J Immunol. 32:3071-3081.

33. Guermonprez, P., Khelef, N., Blouin, E., Rieu, P., Ricciardi-Castagnoli, P., Guiso, N., Ladant, D., and Leclerc, C. 2001. The Adenylate Cyclase Toxin of Bordetella pertussis Binds to Target Cells via the alpha(M)beta(2) Integrin (CD11b/CD18). J Exp Med. 193:1035-1044.

34. Guilloux, Y., Lucas, S., Brichard, V. G., Van Pel, A., Viret, C., De Plaen, E., Brasseur, F., Lethe, B., Jotereau, F., and Boon, T. 1996. A peptide recognized by human cytolytic T lymphocytes on HLA-A2 melanomas is encoded by an intron sequence of the N-acetylglucosaminyltransferase V gene. J Exp Med 183:1173.

35. Haicheur, N., E. Bismuth, S. Bosset, O. Adotevi, G. Warnier, V. Lacabanne, A. Regnault, C. Desaymard, S. Amigorena, P. Ricciardi-Castagnoli, B. Goud, W. H. Fridman, L. Johannes, and E. Tartour. 2000. The B subunit of Shiga toxin fused to a tumor antigen elicits CTL and targets dendritic cells to allow MHC class I-restricted presentation of peptides derived from exogenous antigens. J Immunol. 165:3301.

36. Heath, W. R., and F. R. Carbone. 2001. Cross-presentation, dendritic cells, tolerance and immunity. Annu Rev Immunol. 19:47.

37. Hewinson, R. G., H. M. Vordermeier, et al. (2003). “Use of the bovine model of tuberculosis for the development of improved vaccines and diagnostics.” Tuberculosis 83:119-130.

38. Inaba, K., M. Inaba, N. Romani, H. Aya, M. Deguchi, S. Ikehara, S. Muramatsu, and R. M. Steinman. 1992. Generation of large numbers of dendrific cells from mouse bone marrow cultures supplemented with granulocyte/macrophage colony-stimulating factor. J Exp Med. 176:1693.

39. Johnson, J. L., Nyole, S., Okwera, A., Whalen, C. C., Nsubuga, P., Pekovic, V., Huebner, R., Wallis, R. S., Mugyenyi, P. N., Mugerwa, R. D., et al. 1998. Instability of tuberculin and Candida skin test reactivity in HIV-infected Ugandans. The Uganda-Case Western Reserve University Research Collaboration. Am J Respir Crit Care Med 158:1790-1796.

40. Jondal, M., R. Schirmbeck, and J. Reimann. 1996. MHC class I-restricted CTL responses to exogenous antigens. Immunity. 5:295.

41. Jurcevic, S., Hills, A., Pasvol, G., Davidson, R. N., Ivanyi, J., and Wilkinson, R. J. 1996. T cell responses to a mixture of Mycobacterium tuberculosis peptides with complementary HLA-DR binding profiles. Clin Exp Immunol 105:416-421.

42. Kamath, A. T., J. Pooley, M. A. O'Keeffe, D. Vremec, Y. Zhan, A. M. Lew, A. D'Amico, L. Wu, D. F. Tough, and K. Shortman. 2000. The development, maturation, and turnover rate of mouse spleen dendritic cell populations. J Immunol. 165:6762.

43. Karimova, G., C. Fayolle, S. Gmira, A. Ullmann, C. Leclerc, and D. Ladant. 1998. Charge-dependent translocation of Bordetella pertussis adenylate cyclase toxin into eukaryotic cells: implication for the in vivo delivery of CD8(+) T cell epitopes into antigen-presenting cells. Proc Natl Acad Sci USA. 95:12532.

44. Karttunen, J., S. Sanderson, and N. Shastri. 1992. Detection of rare antigen-presenting cells by the lacZ T-cell activation assay suggests an expression cloning strategy for T-cell antigens. Proc Natl Acad Sci USA. 89:6020.

45. Kern, D. E., J. P. Klarnet, M. C. Jensen, and P. D. Greenberg. 1986. Requirement for recognition of class II molecules and processed tumor antigen for optimal generation of syngeneic tumor-specific class I-restricted CTL. J Immunol. 136:4303.

46. Khelef, N., Gounon, P., and Guiso, N. 2001. Internalization of Bordetella pertussis adenylate cyclase-haemolysin into endocytic vesicles contributes to macrophage cytotoxicity. Cell Microbiol 3:721-730.

47. Krebs, J. R. (1997). Bovine Tuberculosis in cattle and badgers. London, Ministry of Agriculture, Fisheries and Food Publications, London, UK.

48. Krizanova, O., F. Ciampor, and P. Veber. 1982. Influence of chlorpromazine on the replication of influenza virus in chick embryo cells. Acta Virol 26:209.

49. Ladant, D., and A. Ullmann. 1999. Bordatella pertussis adenylate cyclase: a toxin with multiple talents. Trends Microbiol 7:172.

50. Lalvani, A., Brookes, R., Wilkinson, R. J., Malin, A. S., Pathan, A. A., Andersen, P., Dockrell, H. M., Pasvol, G., and Hill, A. V. S. 1998. Human cytolytic and Interferon-g secreting CD8+ T lymphocytes specific for Mycobacterium tuberculosis. Proc Nat Acad Sci USA 95:270-275.

51. Lalvani, A., Pathan, A. A., McShane, H., Wilkinson, R. J., Latif, M., Conlon, C. P., Pasvol, G., and Hill, A. V. 2001. Rapid detection of M. tuberculosis infection by enumeration of antigen-specific T cells. Am J Resp Crit Care Med 15:824-828.

52. Larkin, J. M., M. S. Brown, J. L. Goldstein, and R. G. Anderson. 1983. Depletion of intracellular potassium arrests coated pit formation and receptor-mediated endocytosis in fibroblasts. Cell 33:273.

53. Lich, J. D., J. F. Elliott, and J. S. Blum. 2000. Cytoplasmic Processing Is a Prerequisite for Presentation of an Endogenous Antigen by Major Histocompatibility Complex Class II Proteins. J Exp Med. 191:1513.

54. Lo-Man, R., J. P. Langeveld, E. Deriaud, M. Jehanno, M. Rojas, J. M. Clement, R. H. Meloen, M. Hofnung, and C. Leclerc. 2000. Extending the CD4(+) T-cell epitope specificity of the Th1 immune response to an antigen using a Salmonella enterica serovar typhimurium delivery vehicle. Infect Immun. 68:3079.

55. Loucka, J., Schlecht, G., Vodolanova, J., Leclerc, C., and Sebo, P. 2002. Delivery of a MalE CD4(+)-T-cell epitope into the major histocompatibility complex class II antigen presentation pathway by Bordetella pertussis adenylate cyclase. Infect Immun. 70:1002-1005.

56. Madshus, I. H., K. Sandvig, S. Olsnes, and B. van Deurs. 1987. Effect of reduced endocytosis induced by hypotonic shock and potassium depletion on the infection of Hep 2 cells by picornaviruses. J Cell Physiol 131:14.

57. Mahairas, G. G., Sabo, P. J., Hickey, M. J., Singh, D. C., and Stover, C. K. 1996. Molecular analysis of genetic differences between Mycobacterium bovis BCG and virulent M. bovis. J Bacteriol 178:1274-1282.

58. Manickasingham, S. P., A. D. Edwards, O. Schultz, and C. Reis e Sousa. 2003. The ability of murine dendritic cell subsets to direct T helper cell differentiation is dependent on microbial signals. Eur J Immunol. 33:101.

59. Mizuochi, T., S. T. Yee, M. Kasai, T. Kakiuchi, D. Muno, and E. Kominami. 1994. Both cathepsin B and cathepsin D are necessary for processing of ovalbumin as well as for degradation of class II MHC invariant chain. Immunol Lett 43:189.

60. Moron, G., P. Rueda, I. Casal, and C. Leclerc. 2002. CD8alpha-CD11b+ dendritic cells present exogenous virus-like particles to CD8+ T cells and subsequently express CD8alpha and CD205 molecules. J Exp Med 195:1233.

61. Mukherjee, P., A. Dani, S. Bhatia, N. Singh, A. Y. Rudensky, A. George, V. Bal, S. Mayor, and S. Rath. 2001. Efficient presentation of both cytosolic and endogenous transmembrane protein antigens on MHC class II is dependent on cytoplasmic proteolysis. J Immunol. 167:2632.

62. Osicka, R., Osickova, A., Basar, T., Guermonprez, P., Rojas, M., Leclerc, C., and Sebo, P. 2000. Delivery of CD8(+) T-cell epitopes into major histocompatibility complex class I antigen presentation pathway by Bordetella pertussis adenylate cyclase: delineation of cell invasive structures and permissive insertion sites. Infect Immun 68:247-256.

63. Osicka, R., A. Osickova, T. Basar, P. Guermonprez, M. Rojas, C. Leclerc, and P. Sebo. 1999. Delivery of CD8⁺ T cell epitopes into major histocompatibility complex class I antigen presentation pathway by Bordetella pertussis adenylate cyclase: delineation of cell invasive structures and permissive insertion sites. Infect. Immun. 68:247-256.

64. Pardoll, D. M., and S. L. Topalian. 1998. The role of CD4+ T cell responses in antitumor immunity. Current Opinion in Immunology. 10:588.

65. Pelham, H. R. 1991. Multiple targets for brefeldin A. Cell 67:449.

66. Pollock, J. M., D. A. Pollock, et al. (1996). “Dynamic changes in circulating and antigen-responsive T-cell subpopulations post-Mycobacterium bovis infection in cattle.” Immunology 87(2): 236-41.

67. Pym, A. S., P. Brodin, R. Brosch, M. Huerre, and S. T. Cole. 2002. Loss of RD1 contributed to the attenuation of the live tuberculosis vaccines Mycobacterium bovis BCG and Mycobacterium microti. Mol. Microbiol. 46:709.

68. Pym, A. S., P. Brodin, L. Majlessi, R. Brosch, C. Demangel, A. Rawkins, M. Huerre, C. Leclerc and S. T. Cole. 2003. A recombinant BCG vaccine exporting ESAT-6 via a dedicated secretion apparatus confers enhanced protection against tuberculosis in animal models. Nat Med. 14:533.

69. Reimann, J., and R. Schirmbeck. 1999. Alternative pathways for processing exogenous and endogenous antigens that can generate peptides for MHC class I-restricted presentation. Immunol Rev. 172:131.

70. Rock, K. L., and A. L. Goldberg. 1999. Degradation of cell proteins and the generation of MHC class I-presented peptides. Annu Rev Immunol. 17.739.

71. Rock, K. L., C. Gramm, L. Rothstein, K. Clark, R. Stein, L. Dick, D. Hwang, and A. L. Goldberg. 1994. Inhibitors of the proteasome block the degradation of most cell proteins and the generation of peptides presented on MHC class I molecules. Cell 78:761.

72. Rudensky, A., P. Preston-Hurlburt, S. C. Hong, A. Barlow, and C. A. Janeway, Jr. 1991. Sequence analysis of peptides bound to MHC class II molecules. Nature. 353:622.

73. Ruedl, C., and M. F. Bachmann. 1999. CTL priming by CD8(+) and CD8(−) dendritic cells in vivo. Eur J Immunol. 29:3762.

74. Saron, M. F., Fayolle, C., Sebo, P., Ladarit, D., Ullmann, A., and Leclerc, C. 1997. Anti-viral protection conferred by recombinant adenylate cyclase toxins from Bordetella pertussis carrying a CD8+ T cell epitope from lymphocytic choriomeningitis virus. Proc Natl Acad Sci USA 94:3314-3319.

75. Schlecht, G., C. Leclerc, and G. Dadaglio. 2001. Induction of CTL and nonpolarized Th cell responses by CD8alpha(+) and CD8alpha(−) dendritic cells. J Immunol. 167:4215.

76. Schnell, S., J. W. Young, A. N. Houghton, and M. Sadelain. 2000. Retrovirally transduced mouse dendritic cells require CD4+ T cell help to elicit antitumor immunity: implications for the clinical use of dendritic cells. J Immunol. 164:1243.

77. Sebo, P., P. Glaser, H. Sakamoto, and A. Ullmann. 1991. High-level synthesis of active adenylate cyclase toxin of Bordetella pertussis in a reconstructed Escherichia coli system. Gene. 104:19.

78. Sebo, P., Fayolle, C., d'Andria, O., Ladant, D., Leclerc, C., and Ullmann, A. 1995. Cell-invasive activity of epitope-tagged adenylate cyclase of Bordetella pertussis allows in vitro presentation of a foreign epitope to CD8+ cytotoxic T cells. Infect Immun 63:3851-3857.

79. Skipper, J. C., Hendrickson, R. C., Gulden, P. H., Brichard, V., Van Pel, A., Chen, Y., Shabanowitz, J., Wolfel, T., Slingluff, C. L., Jr., Boon, T., Hunt, D. F., and Engelhard, V. H. 1996. An HLA-A2-restricted tyrosinase antigen on melanoma cells results from posttranslational modification and suggests a novel pathway for processing of membrane proteins. J Exp Med 183:527.

80. Sorensen, A. L., Nagai, S., Houen, G., Andersen, P., and Andersen, A. B. 1995. Purification and characterization of a low-molecular-mass T-cell antigen secreted by Mycobacterium tuberculosis. Infect Immun 63:1710-1717.

81. Toes, R. E., F. Ossendorp, R. Offringa, and C. J. Melief. 1999. CD4 T cells and their role in antitumor immune responses. J Exp Med. 189:753.

82. Umezawa, H. 1976. Structures and activities of protease inhibitors of microbial origin. Methods Enzymol 45:678.

83. Van Kaer, L., P. G. Ashton-Rickardt, H. L. Ploegh, and S. Tonegawa. 1992. TAP1 mutant mice are deficient in antigen presentation, surface class I molecules, and CD4-8+ T cells. Cell. 71:1205.

84. Villadangos, J. 2001. Presentation of antigens by MHC class II molecules: getting the most out of them. Molecular Immunology. 38:329.

85. Vordermeier, H. M., A. Whelan, et al. (2001). “Use of synthetic peptides derived from the antigens ESAT-6 and CFP-10 for differential diagnosis of bovine tuberculosis in cattle.” Clin Diagn Lab Immunol, 8(3): 571-8.

86. Vordermeier, H. M., M. A. Chambers, et al. (2002). “Correlation of ESAT-6-specific gamma interferon production with pathology in cattle following Mycobacterium bovis BCG vaccination against experimental bovine tuberculosis.” Infect Immun 70(6): 3026-32.

87. Vordermeier, H. M., P. C. Cockle, et al. (1999). “Development of diagnostic reagents to differentiate between Mycobacterium bovis BCG vaccination and M. bovis infection in cattle.” Clin Diagn Lab Immunol 6(5): 675-82.

88. Vordermeier, H. M., P. J. Cockle, et al. (2000). “Effective DNA vaccination of cattle with the mycobacterial antigens MPB83 and MPB70 does not compromise the specificity of the comparative intradermal tuberculin skin test.” Vaccine 19(9-10): 1246-55.

89. Wang, L. H., K. G. Rothberg, and R. G. Anderson. 1993. Mis-assembly of clathrin lattices on endosomes reveals a regulatory switch for coated pit formation. J Cell Biol 123:1107.

90. Weinrich Olsen, A., van Pinxteren, L. A., Meng Okkels, L., Birk Rasmussen, P., and Andersen, P. 2001. Protection of mice with a tuberculosis subunit vaccine based on a fusion protein of antigen 85B and ESAT-6. Infect Immun 69:2773-2778.

91. Wilkinson, K. A., Belisle, J. T., Mincek, M., Wilkinson, R. J., and Toossi, Z. 2000. Enhancement of the human T cell response to culture filtrate fractions of Mycobacterium tuberculosis by microspheres. J Immunol Methods 235:1-9.

92. Wilkinson, K. A., Hudecz, F., Vordermeier, H. M., Ivanyl, J., and Wilkinson, R. J. 1999. Enhancement of the T cell response to a mycobacterial peptide by conjugation to synthetic branched polypeptide. Eur J Immunol 29:2788-2796.

93. Wilkinson, R. J., Zhu, X., Wilkinson, K. A., Lalvani, A., Ivanyi, J., Pasvol, G., and Vordermeier, H. M. 1998. 38000 MW Antigen specific MHC Class I restricted Interferon-g secreting CD8+ T cells in healthy contacts of tuberculosis. Immunology 95:585-590.

94. Wolfel, T., Van Pel, A., Brichard, V., Schneider, J., Seliger, B., Meyer zum Buschenfelde, K. H., and Boon, T. 1994. Two tyrosinase nonapeptides recognized on HLA-A2 melanomas by autologous cytolytic T lymphocytes. Eur J Immunol 24:759.

95. Wong, P., and E. G. Pamer. 2003. CD8 T cell responses to infectious pathogens. Annu Rev Immunol. 21:29.

96. Wood, P. R. and J. S. Rothel (1994). “In vitro immunodiagnostic assays for bovine tuberculosis.” Vet. Microbiol. 40: 125-135.

97. Zajac, A. J., K. Murali-Krishna, J. N. Blattman, and R. Ahmed. 1998. Therapeutic vaccination against chronic viral infection: the importance of cooperation between CD4+ and CD8+ T cells. [Review] [53 refs]. Current Opinion in Immunology. 10:444.

98. Dadaglio G. et al, 2003, Recombinant adenylate cyclase toxin of Bordetella pertussis induces cytotoxic T lymphocyte responses against HLA*0201-restricted melanoma epitopes, International Immunology, vol. 15, No. 12, pp. 1423-1430.

1. A method of in vitro diagnosing or immunomonitoring a disease in a mammal or immunomonitoring any other T cell response comprising:
(A) exposing a T cell of the mammal to a recombinant protein, wherein the recombinant protein comprises
(1) a Bordetella CyaA, or a fragment thereof, and
(2) a peptide of an antigen with which T cells of the mammal are suspected to have been previously stimulated; and
(B) detecting a change in activation of the T cell.

2-45. (canceled)
